(19) United States
(12) Patent Application Publication
(10) Pub. No.: US 2017/0213980 A1
(43) Pub. Date: Jul. 27, 2017

(54) MATERIAL FOR ORGANIC ELECTROLUMINESCENT DEVICE AND ORGANIC ELECTROLUMINESCENT DEVICE USING THE SAME

(71) Applicant: SAMSUNG DISPLAY CO., LTD., Yongin-si (KR)

(72) Inventor: Hiromi Nakano, Yokohama (JP)

(21) Appl. No.: 15/246,300

(22) Filed: Aug. 24, 2016

(30) Foreign Application Priority Data
Jan. 22, 2016 (KR) 10-2016-0008194

(51) Int. Cl. H01L 51/00; C07F 7/08; C07D 211/82

(52) U.S. Cl. CPC H01L 51/0061; C07D 211/82; H01L 51/0094; C07F 7/0818; H01L 51/0072; H01L 51/0073; H01L 51/5206

(57) ABSTRACT

An organic electroluminescent device including an anode, a cathode, and a plurality of organic layers between the anode and the cathode, wherein at least one layer selected from the organic layers includes a material for an organic EL device, represented by Formula 1:

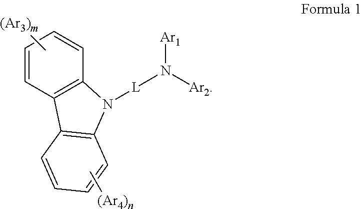

In Formula 1, when position 9 (e.g., the N atom) in a carbazolyl group is combined via L (linker) with a nitrogen atom of an amine group, and at least one selected from position 1 and 8 in the carbazolyl group is substituted with an aryl group, a heteroaryl group, an alkyl group, or a silyl group, the molecule may be non-planar, the volume of the molecule may increase, and the HOMO-LUMO energy gap may increase, thereby increasing or improving the emission efficiency of the organic EL device.

### CROSS-REFERENCE TO RELATED APPLICATION

This application claims priority to and the benefit of Korean Patent Application No. 10-2016-0008194, filed on Jan. 22, 2016 in the Korean Intellectual Property Office, the entire content of which is incorporated herein by reference.

### BACKGROUND

Recently, development of organic electroluminescent (EL) displays as image displays has been actively conducted. An organic EL display is a self-luminescent display that displays an image by emitting light from a luminescent material including an organic compound via the recombination of holes and electrons, which are injected from an anode and a cathode, respectively, into an emission layer. An organic EL display is different from a liquid crystal display.

An example organic EL device may include an anode, a hole transport layer on the anode, an emission layer on the hole transport layer, an electron transport layer on the emission layer, and a cathode on the electron transport layer. Holes may be injected from the anode, and the injected holes may move through the hole transport layer into the emission layer. Electrons may be injected from the cathode, and the injected electrons may move through the electron transport layer into the emission layer. The holes and the electrons injected into the emission layer may recombine to produce excitons in the emission layer. The organic EL device may emit light via radiation deactivation (e.g., radiative decay) of the excitons. Embodiments of the configuration of the organic EL device are not limited to the above-described configuration, however, and one or more suitable modifications may be possible.

An increase in the emission efficiency of organic EL devices is required for suitable application of organic EL devices in a display. For example, the driving voltage of an organic EL device may be high, and the emission efficiency may be insufficient in the blue and green emission regions when compared to the red emission region. Normalization, stabilization, increasing the durability, etc. of a hole transport layer, etc. have been examined as strategies to attain high emission efficiencies in organic EL devices.

A compound obtained by introducing various dibenzoheterole groups to an amine derivative is available in the related art and has been explored as a hole transport material (used in the hole transport layer) to enable the device to attain high efficiency and long life. For example, an amine derivative obtained by introducing a carbazolyl group to a tertiary amine is available in the related art. However, an organic EL device using this amine derivative may not have sufficient emission efficiency. Accordingly, development of an organic EL device having further improved emission efficiency is still needed.

### SUMMARY

One or more aspects of example embodiments of the present disclosure are directed toward a material for an organic electroluminescent device and an organic electroluminescent device using the same; specifically, to a hole transport material for an organic electroluminescent device having high efficiency, and an organic electroluminescent device using the same.

One or more example embodiments of the present disclosure provide a material for an organic EL device having high emission efficiency, and an organic EL device using the same.

One or more example embodiments of the present disclosure also provide a material for an organic EL device that has high emission efficiency in the blue and green emission regions and is used in at least one laminated layer between an emission layer and an anode, and an organic EL device including the same.

An example embodiment of the present disclosure provides a material for an organic EL device, represented by Formula 1:

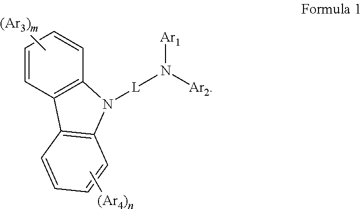

In Formula 1, Ar₁ and Ar₂ may each independently be a substituted or unsubstituted aryl group having 6 to 30 carbon atoms for forming a ring, or a substituted or unsubstituted heteroaryl group having 1 to 30 carbon atoms for forming a ring; Ar₃ and Ar₄ may each independently be a substituted or unsubstituted aryl group having 6 to 30 carbon atoms for forming a ring, a substituted or unsubstituted heteroaryl group having 5 to 30 carbon atoms for forming a ring, an alkyl group having 1 to 6 carbon atoms, or a silyl group; L may be a substituted or unsubstituted arylene group having 6 to 30 carbon atoms for forming a ring, or a substituted or unsubstituted heteroarylene group having 5 to 30 carbon atoms for forming a ring; m and n may each independently be an integer selected from 0 to 4; m+n≧1; and at least one of Ar₃ or Ar₄ may be substituted in at least one of position 1 or position 8 of a carbazolyl group (e.g., Ar₃ may be substituted at position 1, Ar₄ may be substituted at position 8, or both Ar₃ and Ar₄ may be substituted at position 1 and position 8, respectively, of a carbazolyl group).

In the material for an organic EL device according to an embodiment of the present disclosure, position 9 (e.g., the N atom) of the carbazolyl group may be combined (e.g., coupled) via L (linker) with the nitrogen atom of an amine group, and at least one selected from position 1 and position 8 of the carbazolyl group may be substituted with an aryl group, a heteroaryl group, an alkyl group, or a silyl group, thereby improving the emission efficiency of the organic EL device.

In one embodiment, the relation of m+n=1 may be satisfied in Formula 1.

In the material for an organic EL device according to an embodiment of the present disclosure, one selected from Ar₃ and Ar₄ may be substituted at position 1 or position 8 of the carbazolyl group (e.g., the carbazolyl group may be substituted once, either by Ar₃ at position 1 or by Ar₄ at position 8), and the emission efficiency of the organic EL device may be improved.

In the material for an organic EL device according to an embodiment of the present disclosure, Ar₃ and Ar₄ may each independently be a phenyl group, a naphthyl group, a biphenyl group, a fluoroaryl group, a dibenzofuranyl group, or a silyl group.

In one embodiment, L in Formula 1 may be selected from arylene groups (L-1) to (L-7):

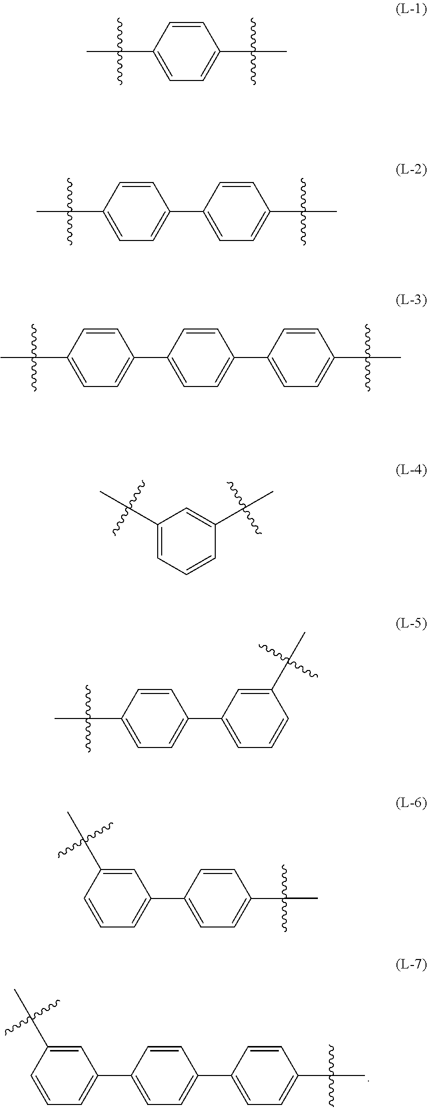

In the material for an organic EL device according to an embodiment of the present disclosure, L may be selected from arylene groups (L-1) to (L-7), and the emission efficiency of the organic EL device may be improved.

In one embodiment, L may be an m-phenylene group.

In the material for an organic EL device according to an embodiment of the present disclosure, L may be an m-phenylene group, and the emission efficiency of the organic EL device may be improved.

In an organic EL device according to an embodiment of the present disclosure, the material for an organic EL device may be included in at least one layer, and high emission efficiency may be attained.

In one embodiment of the present disclosure, an organic EL device includes an anode, a cathode on the anode, and a plurality of organic layers between the anode and the cathode, wherein at least one selected from the plurality of organic layers includes a material for an organic EL device according to an embodiment of the present disclosure.

In one embodiment, the organic EL device may include the material for an organic EL device in at least one layer laminated between an emission layer and the anode.

The organic EL device according to an embodiment of the present disclosure may include the material for an organic EL device in at least one laminated layer between the emission layer and the anode, and high emission efficiency may be attained.

In one embodiment, the organic layer including the material for an organic EL device may be at least one selected from a hole injection layer and a hole transport layer.

The organic EL device according to an embodiment of the present disclosure may include the material for an organic EL device described above in one selected from a hole injection layer and a hole transport layer, and high emission efficiency may be attained.

### BRIEF DESCRIPTION OF THE DRAWINGS

The accompanying drawings are included to enable further understanding of the present disclosure, and are incorporated in and constitute a part of this specification. The drawings illustrate example embodiments of the present disclosure and, together with the description, serve to explain principles of the present disclosure. In the drawings:

FIG. 1 is a schematic diagram illustrating the structure of an organic EL device 100 according to an embodiment of the present disclosure; and

FIG. 2 is a schematic diagram illustrating the structure of an organic EL device 200 according to an embodiment of the present disclosure.

### DETAILED DESCRIPTION

High efficiency in an organic EL device may be accomplished by using a material in which position 9 (e.g., the N atom) in a carbazolyl group is combined (e.g., coupled) via L (linker) with a nitrogen atom of an amine, and least one selected from positions 1 and 8 of the carbazolyl group is substituted with an aryl group, a heteroaryl group, an alkyl group, or a silyl group.

Hereinafter, a material for an organic EL device and an organic EL device according to an embodiment of the present disclosure will be described in more detail with reference to the accompanying drawings. The material for the organic EL device and the organic EL device according to an embodiment of the present disclosure may, however, be embodied in different forms, and should not be construed as being limited to the embodiments set forth herein. In the drawings, elements having substantially the same function will be designated by the same reference numerals, and repeated explanation thereof may not be provided.

In the drawings, the thicknesses of layers, films, panels, regions, etc., may be exaggerated for clarity. It will be understood that when an element such as a layer, film, region, or substrate is referred to as being “on” another element, it can be directly on the other element or intervening element(s) may also be present. In contrast, when an element is referred to as being “directly on” another element, no intervening elements are present.

In the present disclosure,

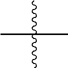

in the formulas denotes that another molecule or group may be connected at that position.

The material for an organic EL device according to an embodiment of the present disclosure may be an amine compound represented by Formula 1:

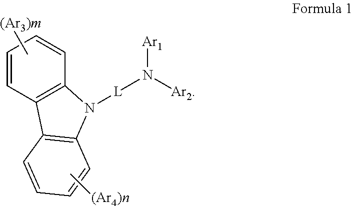

In Formula 1, Ar₁ and Ar₂ may each independently be a substituted or unsubstituted aryl group having 6 to 30 carbon atoms for forming a ring, or a substituted or unsubstituted heteroaryl group having 1 to 30 carbon atoms for forming a ring. Ar₃ and Ar₄ may each independently be a substituted or unsubstituted aryl group having 6 to 30 carbon atoms for forming a ring, a substituted or unsubstituted heteroaryl group having 5 to 30 carbon atoms for forming a ring, an alkyl group having 1 to 6 carbon atoms, or a silyl group, and at least one of Ar₃ or Ar₄ may be substituted in at least one of position 1 or 8 of a carbazolyl group (e.g., Ar₃ may be substituted at position 1, Ar₄ may be substituted at position 8, or both Ar₃ and Ar₄ may be substituted at position 1 and position 8, respectively, of a carbazolyl group). L may be a substituted or unsubstituted arylene group having 6 to 30 carbon atoms for forming a ring, or a substituted or unsubstituted heteroarylene group having 5 to 30 carbon atoms for forming a ring. In some embodiments, m and n may each independently be an integer selected from 0 to 4, and m+n≧1. As used herein, “atoms for forming a ring” may refer to “ring-forming atoms”.

In Formula 1, non-limiting examples of the aryl group having 6 to 30 carbon atoms for forming a ring used as Ar₁ and Ar₂ may include, for example, a phenyl group, a naphthyl group, an anthracenyl group, a phenanthryl group, a biphenyl group, a terphenyl group, a quaterphenyl group, a fluorenyl group, a triphenylene group, a biphenylene group, a pyrenyl group, a benzofluoranthenyl group, a glyceryl group, a phenylnaphthyl group, a naphthylphenyl group, etc.

Non-limiting examples of the heteroaryl group having 1 to 30 carbon atoms for forming a ring and used as Ar₁ and Ar₂ may include, for example, a pyridyl group, a quinolyl group, an isoquinolyl group, a benzofuryl group, a benzothienyl group, an indolyl group, a benzoxazolyl group, a benzothiazolyl group, a quinoxalyl group, a benzimidazolyl group, a dibenzofuryl group, a dibenzothienyl group, a carbazolyl group, etc.

In Formula 2, non-limiting examples of the substituted or unsubstituted aryl group having 6 to 30 carbon atoms for forming a ring and used as Ar₃ and Ar₄ may include a phenyl group, a naphthyl group, an anthracenyl group, a phenanthryl group, a biphenyl group, a terphenyl group, a quaterphenyl group, a fluorenyl group, a triphenylene group, a biphenylene group, a pyrenyl group, a benzofluoranthenyl group, a glyceryl group, a phenylnaphthyl group, a naphthylphenyl group, etc.

Non-limiting examples of the heteroaryl group having 5 to 30 carbon atoms for forming a ring and used as Ar₃ and Ar₄ may include a pyridyl group, a quinolyl group, an isoquinolyl group, a benzofuryl group, a benzothienyl group, an indolyl group, a benzoxazolyl group, a benzothiazolyl group, a quinoxalyl group, a benzimidazolyl group, a dibenzofuranyl group, a dibenzothienyl group, a carbazolyl group, etc.

Non-limiting examples of the alkyl group having 1 to 6 carbon atoms and used as Ar₃ and Ar₄ may include, for example, a methyl group, an ethyl group, a propyl group, an isopropyl group, an n-butyl group, an s-butyl group, an isobutyl group, a t-butyl group, an n-pentyl group, an n-hexyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, etc.

Non-limiting examples of the silyl group used as Ar₃ and Ar₄ may include a trialkylsilyl group, a triarylsilyl group, a monoalkyldiarylsilyl group, a dialkylmonoarylsilyl group, etc.

When Ar₃ and/or Ar₄ are substituted in at least one of position 1 or 8 of a carbazolyl group (e.g., Ar₃ is substituted at position 1, Ar₄ is substituted at position 8, or both Ar₃ and Ar₄ are substituted at position 1 and position 8, respectively, of a carbazolyl group), non-limiting examples of Ar₃ and Ar₄ may include a phenyl group, a naphthyl group, a biphenyl group, a fluoroaryl group, a dibenzofuranyl group, and a silyl group. The number of Ar₃ and Ar₄ groups substituted on the carbazolyl group is not specifically limited; however, in some embodiments, m+n=1, and Ar₃ or Ar₄ may be substituted at position 1 or 8 of the carbazolyl group (e.g., Ar₃ may be substituted at position 1, or Ar₄ may be substituted at position 8).

In Formula 1, non-limiting examples of the arylene group having 6 to 30 carbon atoms for forming a ring and used as L may include a phenylene group, a biphenylene group, a terphenylene group, a naphthylene group, an anthracenyl group, a fluorenylene group, a triphenylene group, a quaterphenylene group, a quinquephenylene group, etc.

Non-limiting examples of the heteroarylene group having 5 to 30 carbon atoms for forming a ring and used as L may include a benzothiazolylene group, a thiophenylene group, a thienothiophenylene group, a thienothienothiophenylene group, a benzothiophenylene group, a benzofuranylene group, a dibenzothiophenylene group, a dibenzofuranylene group, a carbazolyl group, a phenoxazinyl group, etc.

L may be selected from arylene groups (L-1) to (L-7), and in some embodiments, L may be an m-phenylene group.

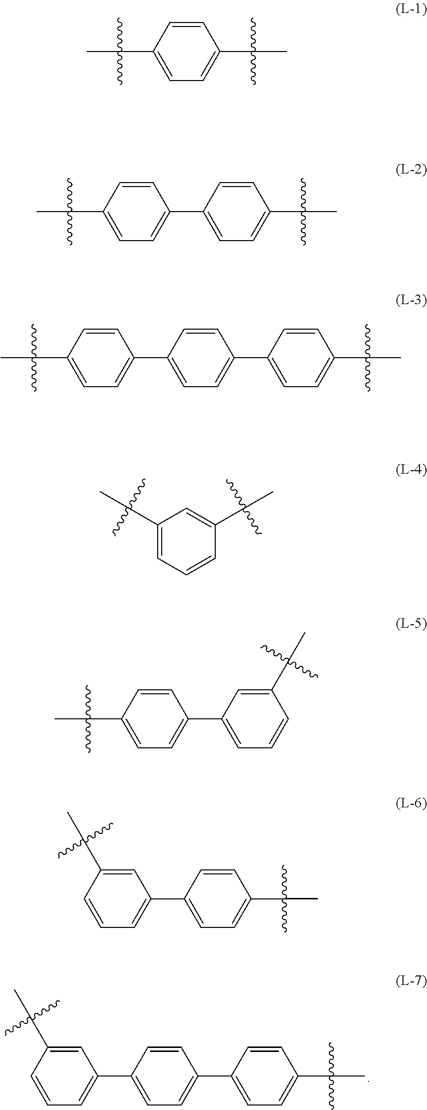

In Formula 1, when Ar₁ to Ar₄ and/or L have a substituent (e.g., are substituted), the substituent may be an alkyl group (such as a methyl group, an ethyl group, a propyl group, a pentyl group, and/or a hexyl group) or an aryl group (such as a phenyl group, a biphenyl group, and/or a naphthyl group). In some embodiments, Ar₁ to Ar₄ and L may each have a plurality of substituents. In some embodiments, adjacent substituents may be connected (e.g., coupled) to form a saturated or unsaturated ring.

In the material for an organic EL device according to an embodiment of the present disclosure as represented by Formula 1, position 9 (e.g., the N atom) in a carbazolyl group may be combined (e.g., coupled) via L (linker) with a nitrogen atom of an amine group, and at least one selected from positions 1 and 8 of the carbazolyl group may be substituted with an aryl group, a heteroaryl group, an alkyl group, or a silyl group. Accordingly, the whole molecule of the amine compound may be distorted (e.g., the molecule as a whole may be non-planar), the volume of the molecule may increase, and the HOMO-LUMO energy gap may increase. Therefore, the emission efficiency of the organic EL device may be improved or increased.

The material for an organic EL device according to an embodiment of the present disclosure may be, for example, a material represented by at least one selected from Compounds 1 to 50:

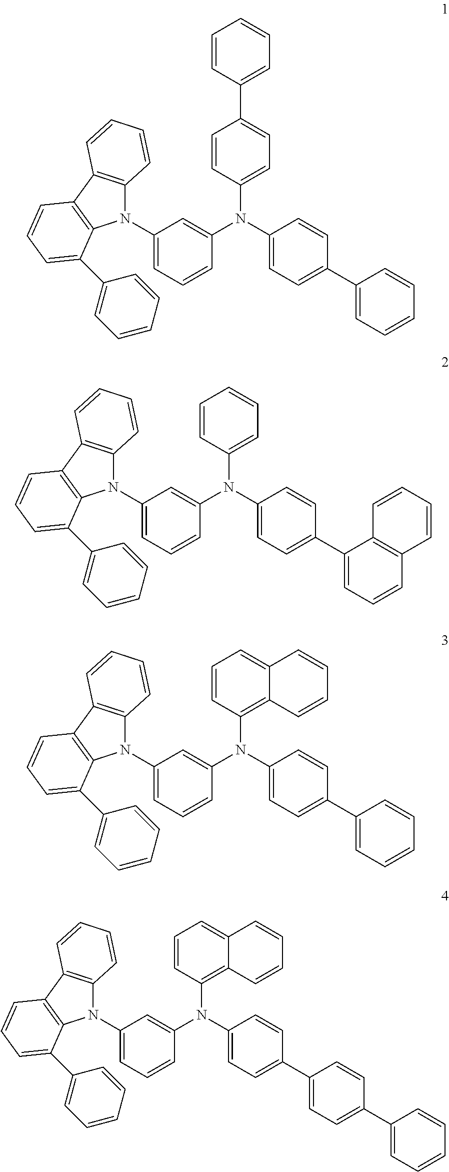

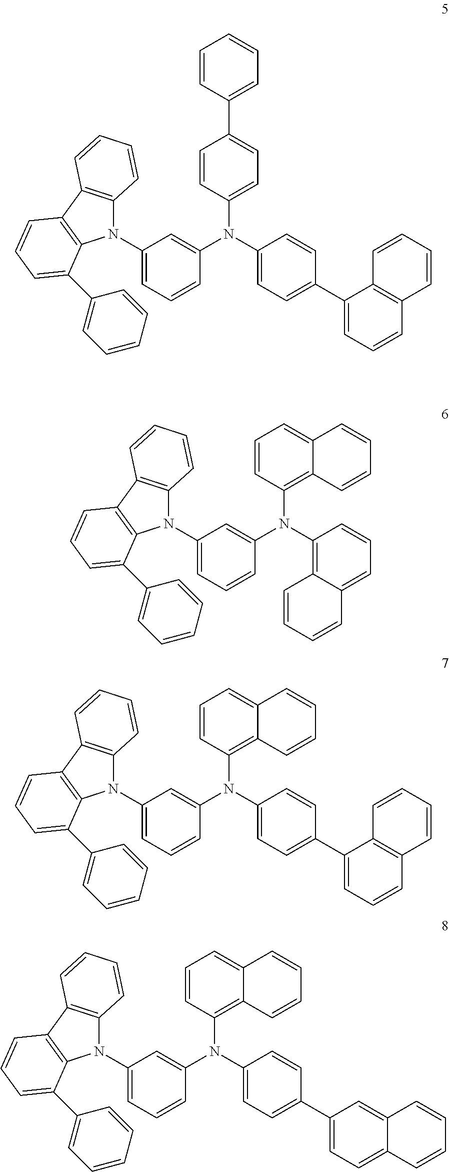 [SMILES: c1ccc(-c2ccc(N(c3ccc(-c4cccc5ccccc45)cc3)c3cccc(-n4c5ccccc5c5cccc(-c6ccccc6)c54)c3)cc2)cc1 ; c1ccc(-c2cccc3c4ccccc4n(-c4cccc(N(c5cccc6ccccc56)c5cccc6ccccc56)c4)c23)cc1 ; c1ccc(-c2cccc3c4ccccc4n(-c4cccc(N(c5ccc(-c6cccc7ccccc67)cc5)c5cccc6ccccc56)c4)c23)cc1 ; c1ccc(-c2cccc3c4ccccc4n(-c4cccc(N(c5ccc(-c6ccc7ccccc7c6)cc5)c5cccc6ccccc56)c4)c23)cc1]

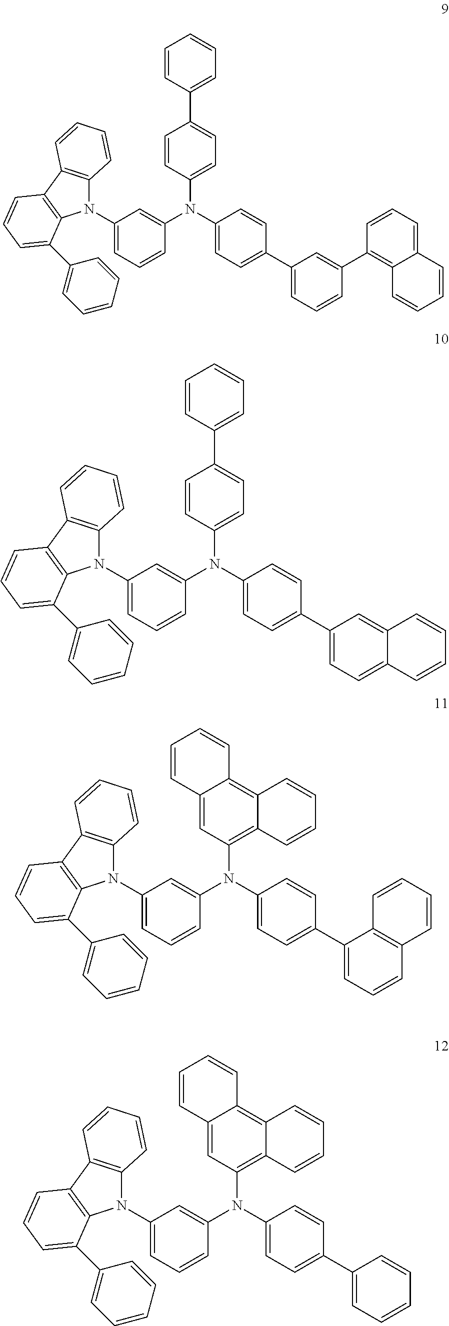 [SMILES: c1ccc(-c2ccc(N(c3ccc(-c4cccc(-c5cccc6ccccc56)c4)cc3)c3cccc(-n4c5ccccc5c5cccc(-c6ccccc6)c54)c3)cc2)cc1 ; c1ccc(-c2ccc(N(c3ccc(-c4ccc5ccccc5c4)cc3)c3cccc(-n4c5ccccc5c5cccc(-c6ccccc6)c54)c3)cc2)cc1 ; c1ccc(-c2cccc3c4ccccc4n(-c4cccc(N(c5ccc(-c6cccc7ccccc67)cc5)c5cc6ccccc6c6ccccc56)c4)c23)cc1 ; c1ccc(-c2ccc(N(c3cccc(-n4c5ccccc5c5cccc(-c6ccccc6)c54)c3)c3cc4ccccc4c4ccccc34)cc2)cc1]

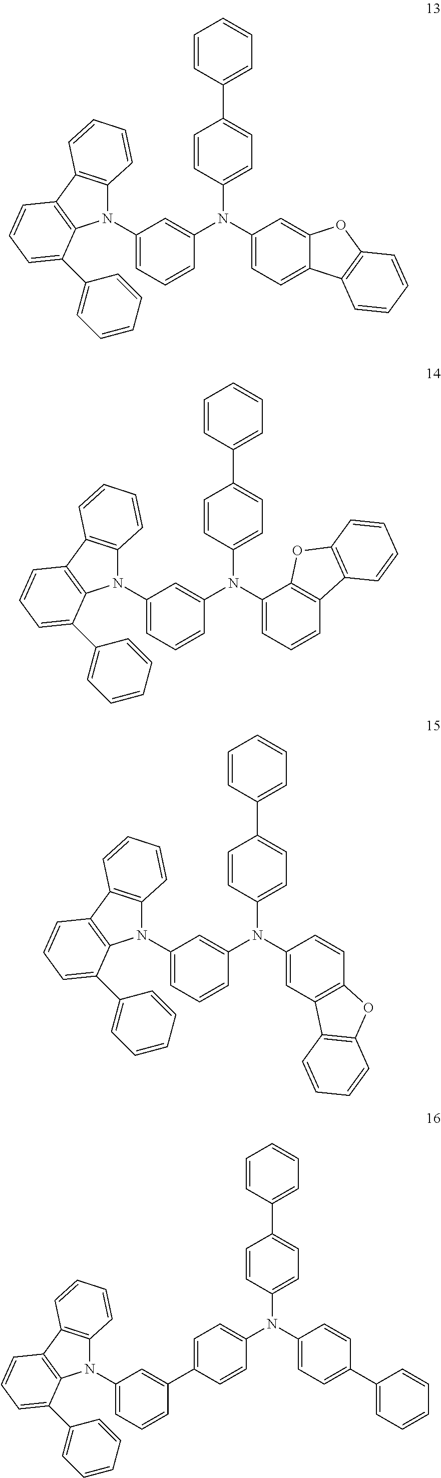 [SMILES: c1ccc(-c2ccc(N(c3cccc(-n4c5ccccc5c5cccc(-c6ccccc6)c54)c3)c3ccc4c(c3)oc3ccccc34)cc2)cc1 ; c1ccc(-c2ccc(N(c3cccc(-n4c5ccccc5c5cccc(-c6ccccc6)c54)c3)c3cccc4c3oc3ccccc34)cc2)cc1 ; c1ccc(-c2ccc(N(c3cccc(-n4c5ccccc5c5cccc(-c6ccccc6)c54)c3)c3ccc4oc5ccccc5c4c3)cc2)cc1 ; c1ccc(-c2ccc(N(c3ccc(-c4ccccc4)cc3)c3ccc(-c4cccc(-n5c6ccccc6c6cccc(-c7ccccc7)c65)c4)cc3)cc2)cc1]

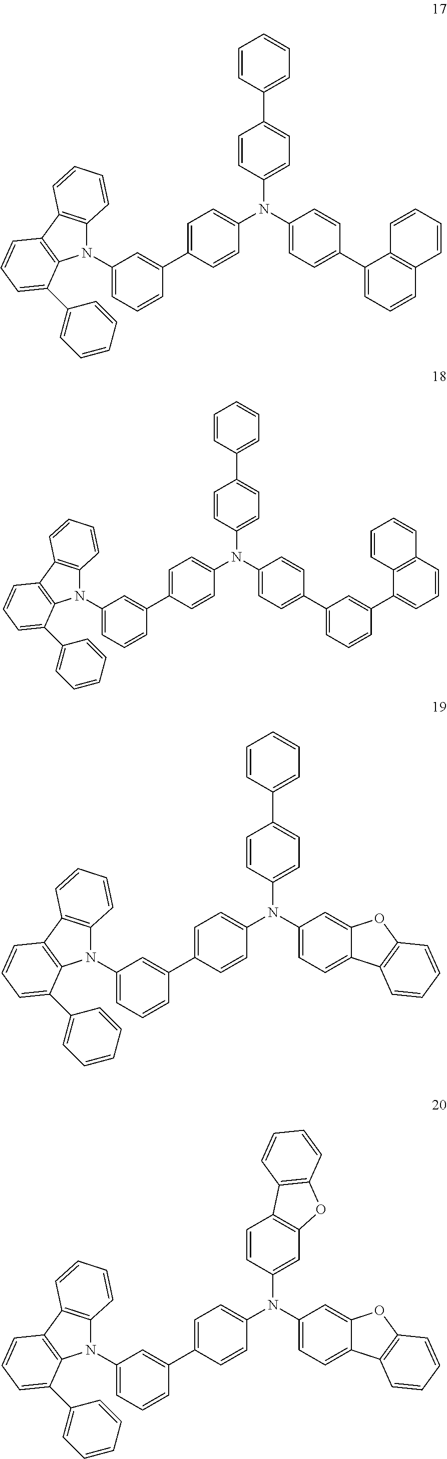 [SMILES: c1ccc(-c2ccc(N(c3ccc(-c4cccc(-n5c6ccccc6c6cccc(-c7ccccc7)c65)c4)cc3)c3ccc(-c4cccc5ccccc45)cc3)cc2)cc1 ; c1ccc(-c2ccc(N(c3ccc(-c4cccc(-c5cccc6ccccc56)c4)cc3)c3ccc(-c4cccc(-n5c6ccccc6c6cccc(-c7ccccc7)c65)c4)cc3)cc2)cc1 ; c1ccc(-c2ccc(N(c3ccc(-c4cccc(-n5c6ccccc6c6cccc(-c7ccccc7)c65)c4)cc3)c3ccc4c(c3)oc3ccccc34)cc2)cc1 ; c1ccc(-c2cccc3c4ccccc4n(-c4cccc(-c5ccc(N(c6ccc7c(c6)oc6ccccc67)c6ccc7c(c6)oc6ccccc67)cc5)c4)c23)cc1]

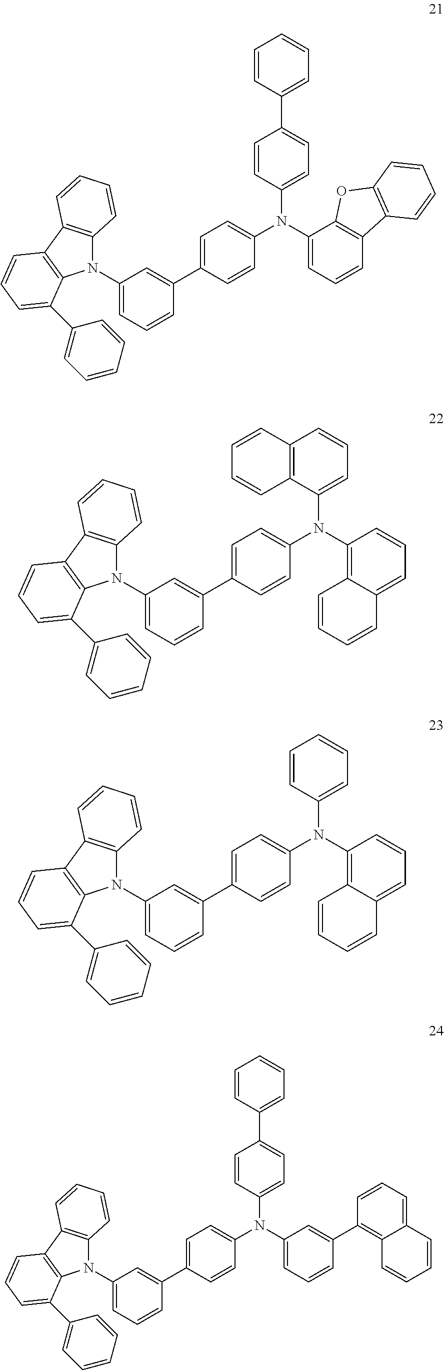 [SMILES: c1ccc(-c2ccc(N(c3ccc(-c4cccc(-n5c6ccccc6c6cccc(-c7ccccc7)c65)c4)cc3)c3cccc4c3oc3ccccc34)cc2)cc1 ; c1ccc(-c2cccc3c4ccccc4n(-c4cccc(-c5ccc(N(c6cccc7ccccc67)c6cccc7ccccc67)cc5)c4)c23)cc1 ; c1ccc(-c2cccc3c4ccccc4n(-c4cccc(-c5ccc(N(c6ccccc6)c6cccc7ccccc67)cc5)c4)c23)cc1 ; c1ccc(-c2ccc(N(c3ccc(-c4cccc(-n5c6ccccc6c6cccc(-c7ccccc7)c65)c4)cc3)c3cccc(-c4cccc5ccccc45)c3)cc2)cc1]

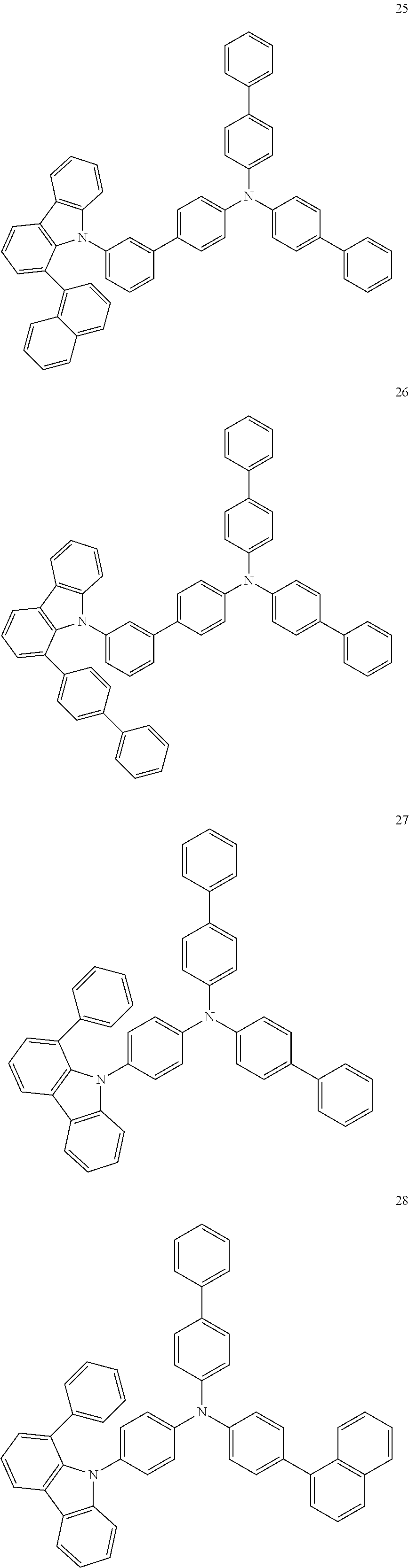 [SMILES: c1ccc(-c2ccc(N(c3ccc(-c4ccccc4)cc3)c3ccc(-c4cccc(-n5c6ccccc6c6cccc(-c7cccc8ccccc78)c65)c4)cc3)cc2)cc1 ; c1ccc(-c2ccc(-c3cccc4c5ccccc5n(-c5cccc(-c6ccc(N(c7ccc(-c8ccccc8)cc7)c7ccc(-c8ccccc8)cc7)cc6)c5)c34)cc2)cc1 ; c1ccc(-c2ccc(N(c3ccc(-c4ccccc4)cc3)c3ccc(-n4c5ccccc5c5cccc(-c6ccccc6)c54)cc3)cc2)cc1 ; c1ccc(-c2ccc(N(c3ccc(-c4cccc5ccccc45)cc3)c3ccc(-n4c5ccccc5c5cccc(-c6ccccc6)c54)cc3)cc2)cc1]

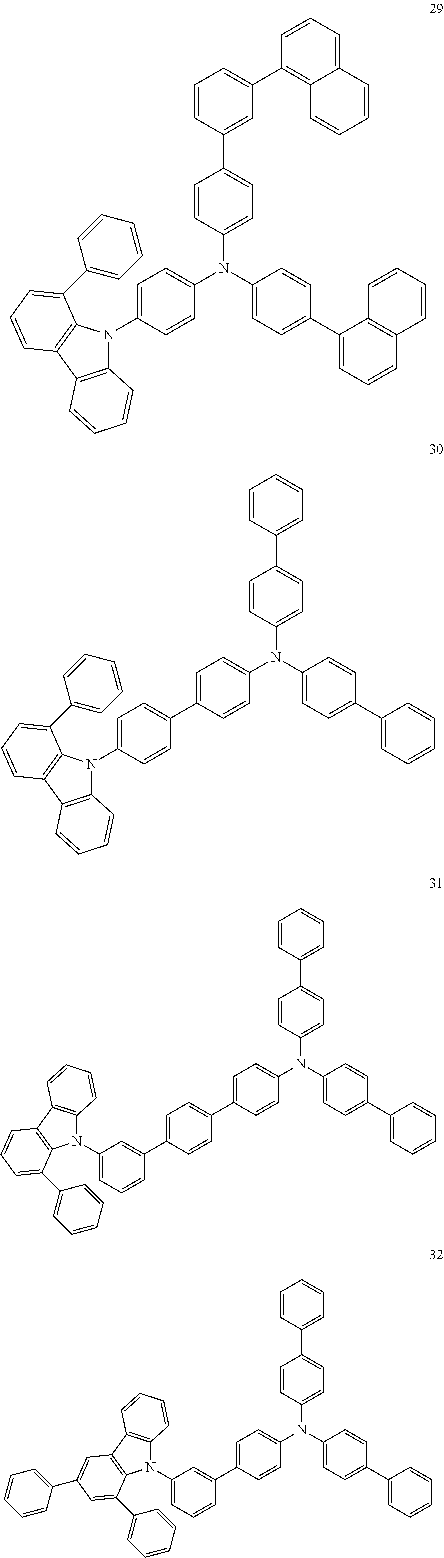 [SMILES: c1ccc(-c2cccc3c4ccccc4n(-c4ccc(N(c5ccc(-c6cccc(-c7cccc8ccccc78)c6)cc5)c5ccc(-c6cccc7ccccc67)cc5)cc4)c23)cc1 ; c1ccc(-c2ccc(N(c3ccc(-c4ccccc4)cc3)c3ccc(-c4ccc(-n5c6ccccc6c6cccc(-c7ccccc7)c65)cc4)cc3)cc2)cc1 ; c1ccc(-c2ccc(N(c3ccc(-c4ccccc4)cc3)c3ccc(-c4ccc(-c5cccc(-n6c7ccccc7c7cccc(-c8ccccc8)c76)c5)cc4)cc3)cc2)cc1 ; c1ccc(-c2ccc(N(c3ccc(-c4ccccc4)cc3)c3ccc(-c4cccc(-n5c6ccccc6c6cc(-c7ccccc7)cc(-c7ccccc7)c65)c4)cc3)cc2)cc1]

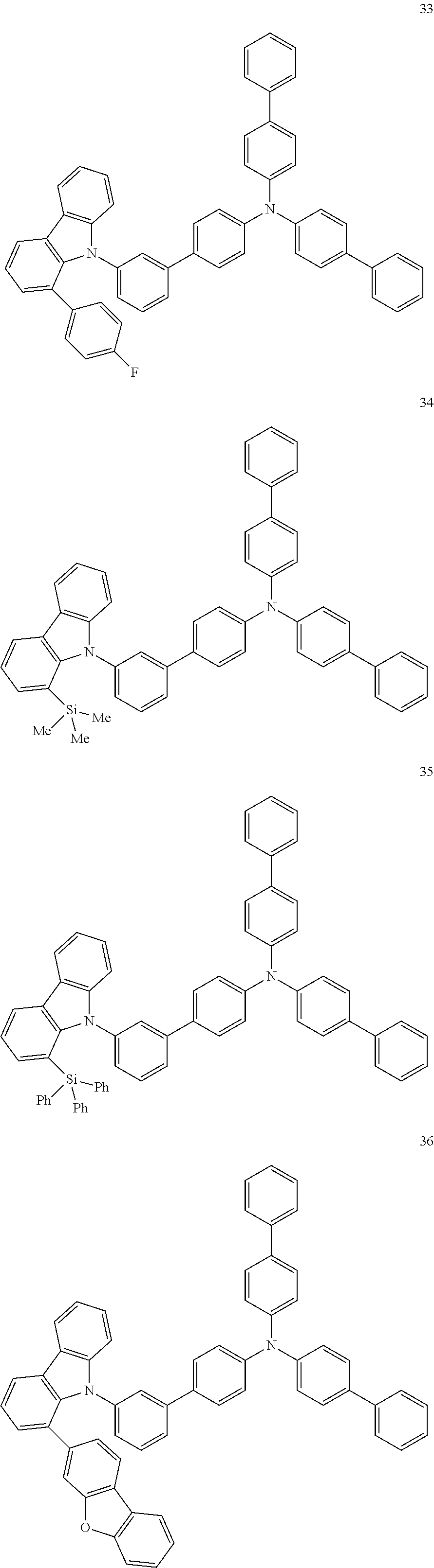 [SMILES: Fc1ccc(-c2cccc3c4ccccc4n(-c4cccc(-c5ccc(N(c6ccc(-c7ccccc7)cc6)c6ccc(-c7ccccc7)cc6)cc5)c4)c23)cc1 ; C[Si](C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2ccc(N(c3ccc(-c4ccccc4)cc3)c3ccc(-c4cccc(-n5c6ccccc6c6cccc([Si](c7ccccc7)(c7ccccc7)c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8c(c7)oc7ccccc78)c65)c4)cc3)cc2)cc1]

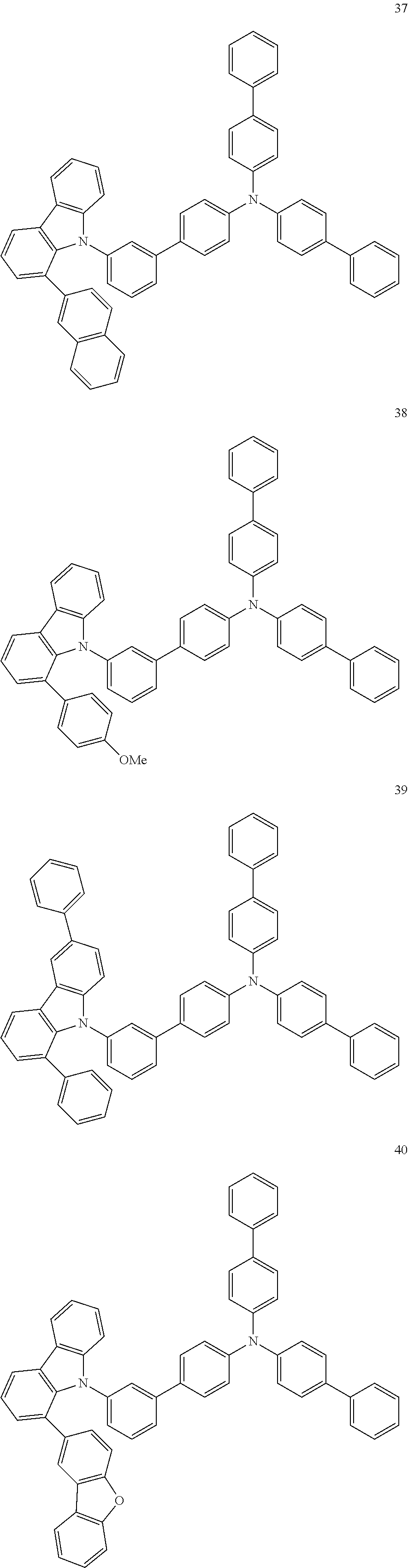 [SMILES: c1ccc(-c2ccc(N(c3ccc(-c4ccccc4)cc3)c3ccc(-c4cccc(-n5c6ccccc6c6cccc(-c7ccc8ccccc8c7)c65)c4)cc3)cc2)cc1 ; COc1ccc(-c2cccc3c4ccccc4n(-c4cccc(-c5ccc(N(c6ccc(-c7ccccc7)cc6)c6ccc(-c7ccccc7)cc6)cc5)c4)c23)cc1 ; c1ccc(-c2ccc(N(c3ccc(-c4ccccc4)cc3)c3ccc(-c4cccc(-n5c6ccc(-c7ccccc7)cc6c6cccc(-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8oc9ccccc9c8c7)c65)c4)cc3)cc2)cc1]

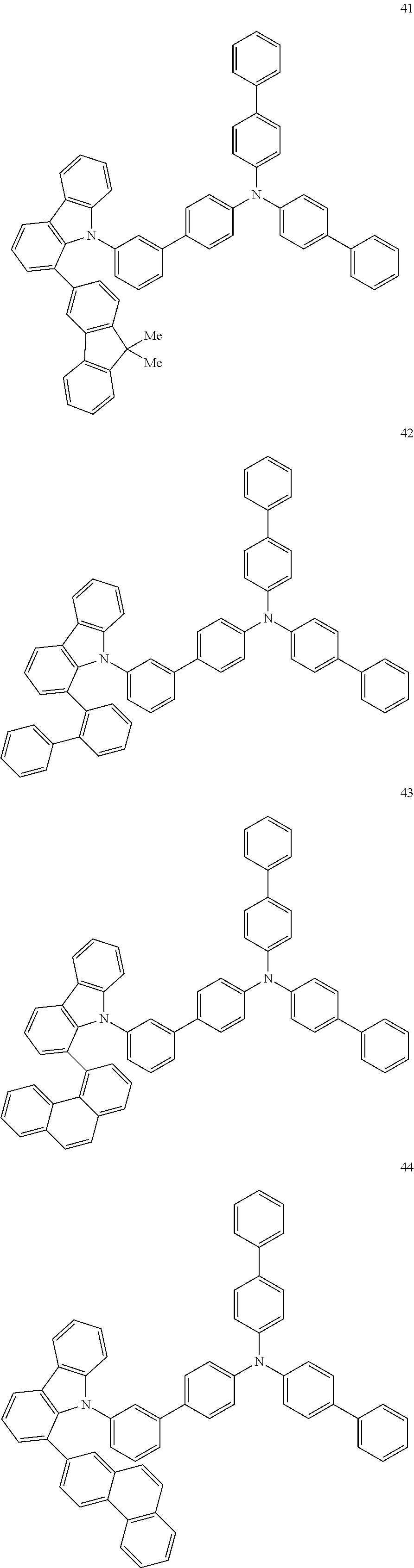 [SMILES: CC1(C)c2ccccc2-c2cc(-c3cccc4c5ccccc5n(-c5cccc(-c6ccc(N(c7ccc(-c8ccccc8)cc7)c7ccc(-c8ccccc8)cc7)cc6)c5)c34)ccc21 ; c1ccc(-c2ccc(N(c3ccc(-c4ccccc4)cc3)c3ccc(-c4cccc(-n5c6ccccc6c6cccc(-c7ccccc7-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7cccc8ccc9ccccc9c78)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8c(ccc9ccccc98)c7)c65)c4)cc3)cc2)cc1]

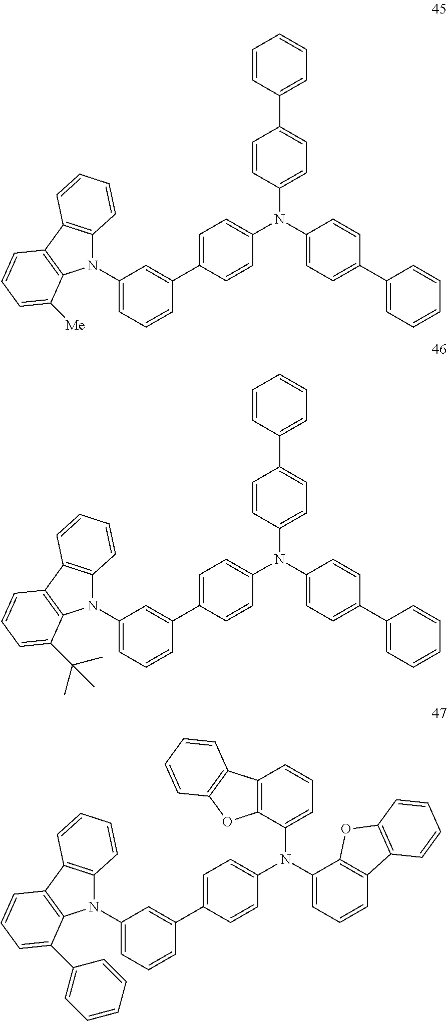 [SMILES: Cc1cccc2c3ccccc3n(-c3cccc(-c4ccc(N(c5ccc(-c6ccccc6)cc5)c5ccc(-c6ccccc6)cc5)cc4)c3)c12 ; CC(C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2cccc3c4ccccc4n(-c4cccc(-c5ccc(N(c6cccc7c6oc6ccccc67)c6cccc7c6oc6ccccc67)cc5)c4)c23)cc1]

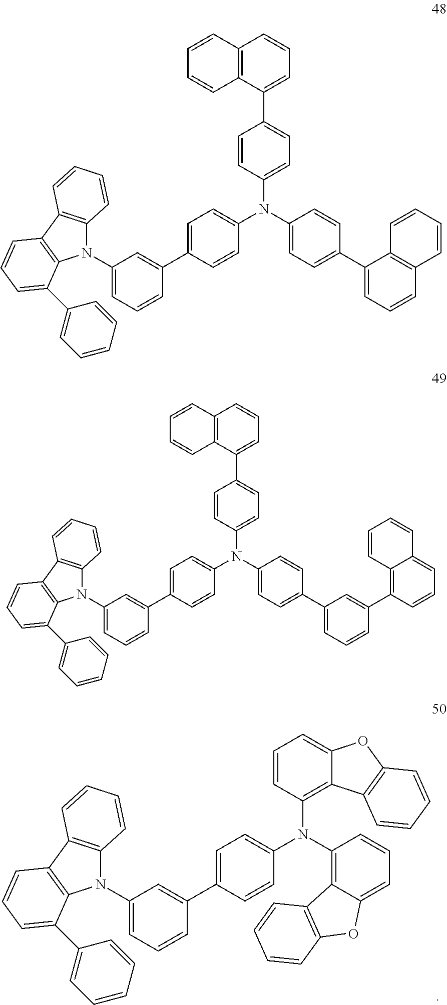 [SMILES: c1ccc(-c2cccc3c4ccccc4n(-c4cccc(-c5ccc(N(c6ccc(-c7cccc8ccccc78)cc6)c6ccc(-c7cccc8ccccc78)cc6)cc5)c4)c23)cc1 ; c1ccc(-c2cccc3c4ccccc4n(-c4cccc(-c5ccc(N(c6ccc(-c7cccc(-c8cccc9ccccc89)c7)cc6)c6ccc(-c7cccc8ccccc78)cc6)cc5)c4)c23)cc1 ; c1ccc(-c2cccc3c4ccccc4n(-c4cccc(-c5ccc(N(c6cccc7oc8ccccc8c67)c6cccc7oc8ccccc8c67)cc5)c4)c23)cc1]

The material for an organic EL device according to an embodiment of the present disclosure may be included in at least one layer selected from a plurality of organic layers forming the organic EL device. For example, the material may be included in at least one layer selected from the laminated layers between an emission layer and an anode in an organic EL device.

As described above, in the material for an organic EL device according to an embodiment of the present disclosure, position 9 (e.g., the N atom) in a carbazolyl group may be combined (e.g., coupled) via L (linker) with a nitrogen atom of an amine group, and at least one selected from positions 1 and 8 of the carbazolyl group may be substituted with an aryl group, a heteroaryl group, an alkyl group, or a silyl group. Accordingly, the whole molecule of the amine compound of the present disclosure may be distorted (e.g., the molecule as a whole may be non-planar), the volume of the molecule may increase, and the HOMO-LUMO energy gap may increase. Therefore, the emission efficiency of the organic EL device may be improved.

### Organic EL Device

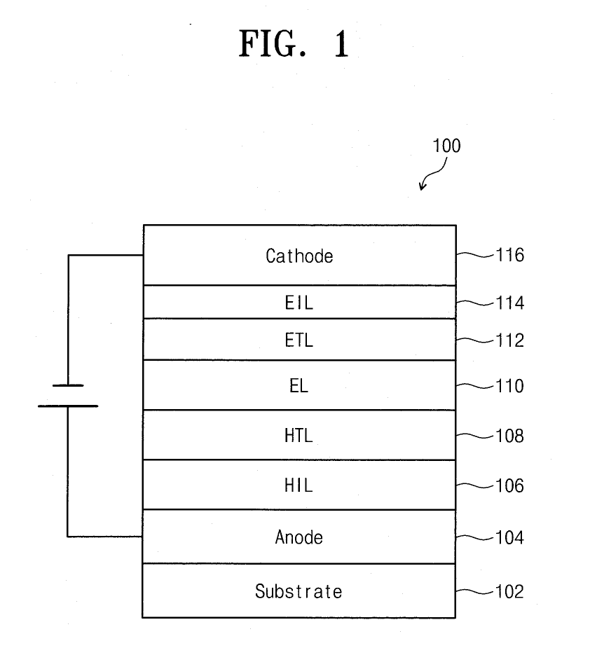

An organic EL device using a material for an organic EL device according to an embodiment of the present disclosure will be explained in more detail. FIG. 1 is a schematic diagram illustrating the structure of an organic EL device 100 according to an embodiment of the present disclosure. The organic EL device 100 may include, for example, a substrate 102, an anode 104, a hole injection layer (HIL) 106, a hole transport layer (HTL) 108, an emission layer (EL) 110, an electron transport layer (ETL) 112, an electron injection layer (EIL) 114, and a cathode 116. In one embodiment, the material for an organic EL device according to the present disclosure may be used in at least one layer selected from the laminated layers between the emission layer and the anode.

An embodiment in which the material for an organic EL device according to the present disclosure is used in the hole transport layer 108 will be explained in more detail.

The substrate 102 may be, for example, a transparent glass substrate, a semiconductor substrate including silicon, a flexible substrate including resin, etc.

The anode 104 may be on the substrate 102 and may be formed using indium tin oxide (In₂O₃—SnO₂:ITO), indium zinc oxide (In₂O₃—ZnO:IZO), etc.

The hole injection layer (HIL) 106 may be provided on the anode 104 using any suitable material at a thickness of about 10 nm to about 150 nm. For example, at least one of triphenylamine-containing polyether ketone (TPAPEK), 4-isopropyl-4′-methyldiphenyliodonium tetrakis(pentafluorophenyl)borate (PPBI), N,N′-diphenyl-N,N′-bis-[4-(phenyl-m-tolyl-amino)-phenyl]-phenyl-4,4′-diamine (DNTPD), a phthalocyanine compound (such as copper phthalocyanine), 4,4′,4″-tris(3-methylphenylphenylamino)triphenylamine (m-MTDATA), N,N′-di(1-naphthyl)-N,N′-diphenylbenzidine (NPB), 4,4′,4″-tris{N,N-diphenylamino}triphenylamine (TDATA), 4,4′,4″-tris(N,N-2-naphthylphenylamino)triphenylamine (2-TNATA), polyaniline/dodecylbenzenesulfonic acid (PANI/DBSA), poly(3,4-ethylenedioxythiophene)/poly(4-styrenesulfonate (PEDOT/PSS), polyaniline/camphor sulfonic acid (PANI/CSA), polyaniline/poly(4-styrenesulfonate (PANI/PSS), etc., may be included.

The hole transport layer (HTL) 108 may be provided on the hole injection layer 106 using the material for an organic EL device according to an embodiment of the present disclosure at a thickness of about 3 nm to about 100 nm. The hole transport layer 108 including the material for an organic EL device according to an embodiment of the present disclosure may be formed, for example, by a vacuum deposition method.

The emission layer (EL) 110 may be formed on the hole transport layer 108 using any suitable host material to a thickness of about 10 nm to about 60 nm. The host material used in the emission layer 110 may include, for example, a condensed polycyclic aromatic derivative, and may be selected from an anthracene derivative, a pyrene derivative, a fluoranthene derivative, a chrysene derivative, a benzoanthracene derivative, and/or a triphenylene derivative. In one embodiment, the emission layer 110 may include the anthracene derivative and/or the pyrene derivative. A compound represented by Formula 2 may be used as the anthracene derivative used in the emission layer 110:

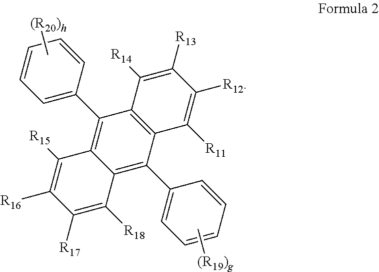

In Formula 2, R₁₁ to R₂₀ may each independently be a substituted or unsubstituted aryl group having 6 to 30 carbon atoms for forming a ring, a substituted or unsubstituted heteroaryl group having 1 to 30 carbon atoms for forming a ring, an alkyl group having 1 to 15 carbon atoms, a silyl group, a halogen atom, a hydrogen atom, or a deuterium atom. In some embodiments, g and h may each independently be an integer selected from 0 to 5. In some embodiments, a plurality of adjacent R₁₁ to R₂₀ groups may be combined (e.g., coupled) to form a saturated or unsaturated ring.

Non-limiting examples of the substituted or unsubstituted heteroaryl group having 1 to 30 carbon atoms for forming a ring used as R₁₁ to R₂₀ may include a benzothiazolyl group, a thiophenyl group, a thienothiophenyl group, a thienothienothiophenyl group, a benzothiophenyl group, a benzofuryl group, a dibenzothiophenyl group, a dibenzofuryl group, a N-aryl carbazolyl group, a N-heteroaryl carbazolyl group, a N-alkyl carbazolyl group, a phenoxazinyl group, a phenothiazyl group, a pyridyl group, a pyrimidyl group, a triazolyl group, a quinolinyl group, a quinoxalyl group, etc.

Non-limiting examples of the alkyl group having 1 to 15 carbon atoms used as R₁₁ to R₂₀ may include a methyl group, an ethyl group, a propyl group, an isopropyl group, an n-butyl group, an s-butyl group, an isobutyl group, a t-butyl group, an n-pentyl group, an n-hexyl group, an n-heptyl group, an n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, an aminomethyl group, a 1-aminoethyl group, a 2-aminoethyl group, a 2-aminoisobutyl group, a 1,2-diaminoethyl group, a 1,3-diaminoisopropyl group, a 2,3-diamino-t-butyl group, a 1,2,3-triaminopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, etc.

The anthracene derivative used in the emission layer 110 of the organic EL device according to an embodiment of the present disclosure may include at least one selected from Compounds a-1 to a-12. However, embodiments of the present disclosure are not limited thereto:

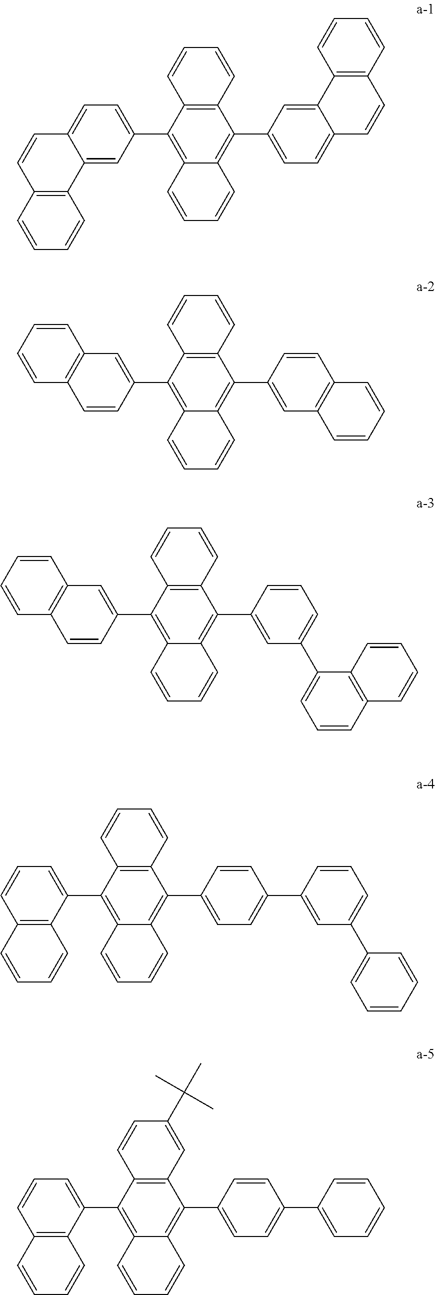

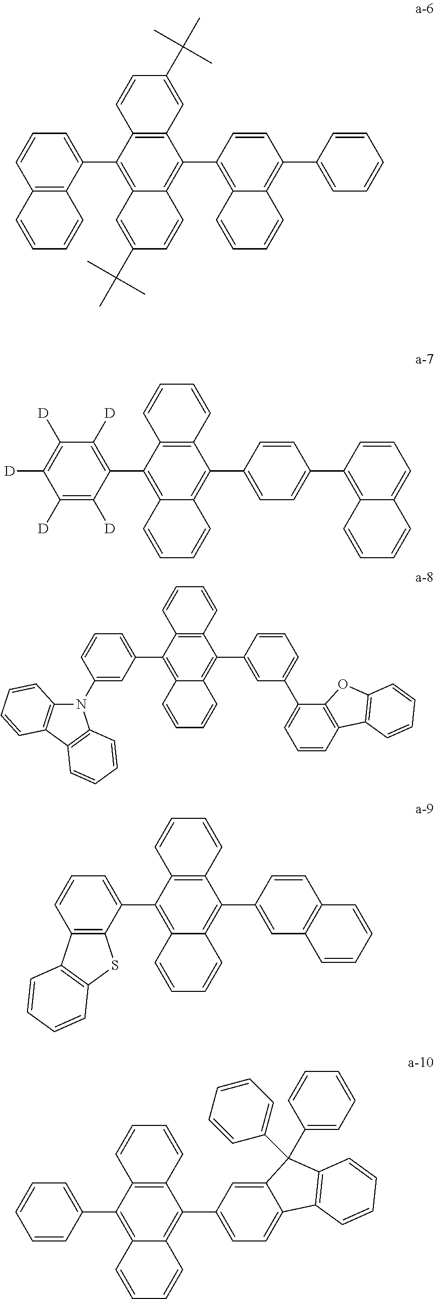

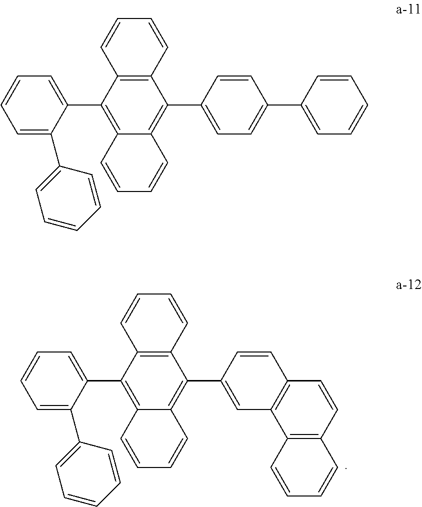

The emission layer 110 may include as a dopant material, for example, styryl derivatives (such as 1,4-bis[2-(3-N-ethylcarbazolyl)vinyl]benzene (BCzVB), 4-(di-p-tolylamino)-4′-[(di-p-tolylamino)styryl]stilbene (DPAVB), and/or N-(4-((E)-2-(6-((E)-4-(diphenylamino)styryl)naphthalene-2-yl)vinyl)phenyl-N-phenylbenzeneamine (N-BDAVBi)), perylene and derivatives thereof (such as 2,5,8,11-tetra-t-butylperylene (TBP)), pyrene and derivatives thereof (such as 1,1-dipyrene, 1,4-dipyrenylbenzene and/or 1,4-bis(N,N-diphenylamino)pyrene), etc., without limitation.

The electron transport layer (ETL) 112 may be formed on the emission layer 110 to a thickness of about 15 nm to about 50 nm using tris(8-hydroxyquinolinato)aluminum (Alq₃) or a material having a nitrogen-containing aromatic ring (for example, a material including a pyridine ring (such as 1,3,5-tri[(3-pyridyl)-phen-3-yl]benzene), a material including a triazine ring (such as 2,4,6-tris(3′-(pyridine-3-yl)biphenyl-3-yl)1,3,5-triazine), and/or a material including an imidazole derivative (such as 2-(4-N-phenylbenzoimidazolyl-1-ylphenyl)-9,10-dinaphthylanthracene)).

The electron injection layer (EIL) 114 may be formed on the electron transport layer 112 to a thickness of about 0.3 nm to about 9 nm using a material including, for example, lithium fluoride (LiF), 8-hydroxyquinolinolato-lithium (LiQ), etc.

The cathode 116 may be on the electron injection layer 114 and may be formed using a metal (such as aluminum (Al), silver (Ag), lithium (Li), magnesium (Mg), calcium (Ca), and/or a mixture thereof), and/or a transparent material (such as ITO and/or indium zinc oxide (In₂O₃—ZnO)).

Each electrode and each layer constituting the organic EL device according to an embodiment of the present disclosure as described above may be formed by selecting an appropriate or suitable layer forming method depending on the material to be used (such as a vacuum deposition method, a sputtering method, and/or one or more suitable coating methods).

In the organic EL device 100 according to an embodiment of the present disclosure, a hole transport layer capable of enabling high efficiency in the organic EL device may be manufactured using the material for an organic EL device according to an embodiment of the present disclosure.

In the organic EL device 100 according to an embodiment of the present disclosure, the material for an organic EL device according to an embodiment of the present disclosure may be used as a material in the hole injection layer. An organic EL device with high efficiency may be manufactured using the material for an organic EL device according to an embodiment of the present disclosure in at least one layer selected from a plurality of organic layers forming the organic EL device.

In some embodiments, the material for an organic EL device according to an embodiment of the present disclosure may be applied to an organic EL display of an active matrix including a thin-film transistor (TFT).

### Preparation Method

The material for an organic EL device according to an embodiment of the present disclosure may be prepared, for example, according to the following synthetic methods.

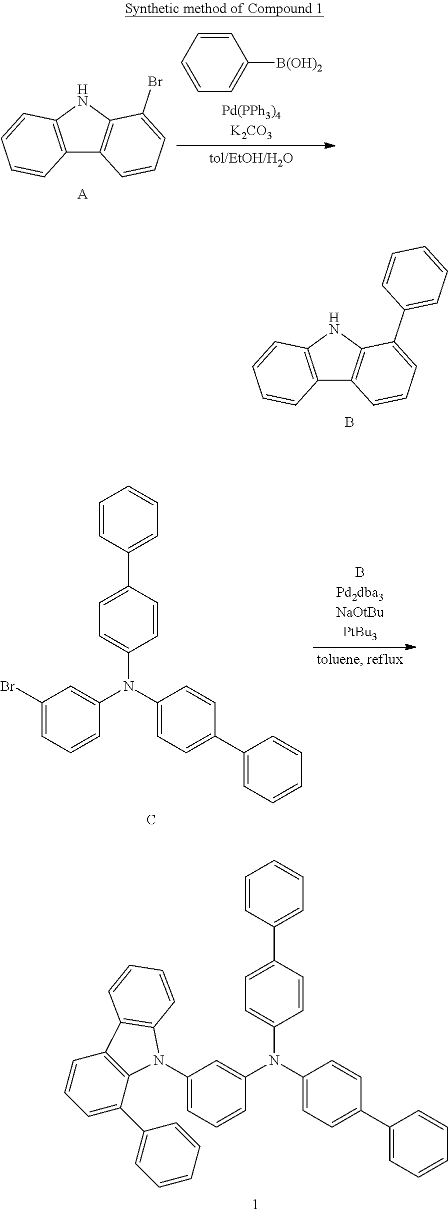

Under an argon atmosphere, 10.0 g of Compound A, 13.3 g of phenyl boronic acid, 3.28 g of tetrakistriphenylphosphine palladium(0) (Pd(PPh₃)₄), and 11.2 g of potassium carbonate (K₂CO₃) were added to a 500 mL, three-necked flask, followed by heating in 200 mL of toluene, 20 mL of ethanol and 20 mL of water at a temperature of 90° C. for 6 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 7.80 g (Yield 79%) of Compound B as a white solid.

Next, under an argon atmosphere, 1.00 g of Compound C, 0.56 g of Compound B, 0.14 g of bis(dibenzylideneacetone)palladium(0) (Pd(dba)₂), 0.15 g of tri-tert-butylphosphine ((t-Bu)₃P), and 0.61 g of sodium tert-butoxide were added to a 100 mL, three-necked flask, followed by refluxing in 25 mL of a toluene solvent for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 1.18 g (Yield 88%) of the target product as a white solid.

The chemical shift values of the target product measured by ¹H-NMR were 8.55 (d, 1H), 8.29 (d, 1H), 8.06 (d, 1H), 7.94 (d, 1H), 7.75-7.73 (m, 4H), 7.55-7.31 (m, 25H). The molecular weight of the target product as measured by fast atom bombardment-mass spectrometry (FAB-MS) was 639. Accordingly, the target product was clearly identified as Compound 1.

In one embodiment, the material for an organic EL device according to an embodiment of the present disclosure may be synthesized, for example, as follows:

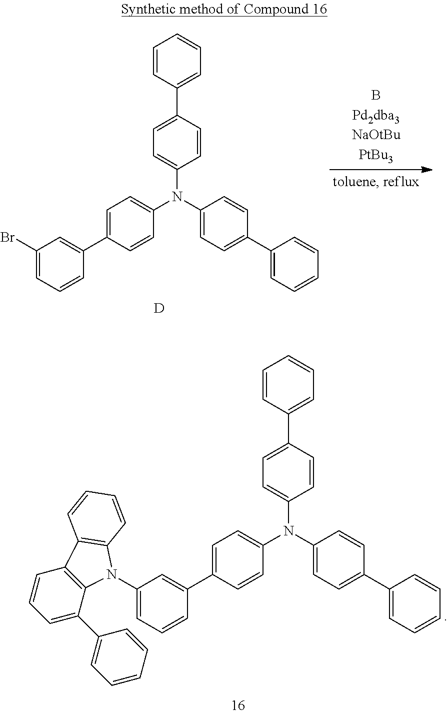

Under an argon atmosphere, 1.50 g of Compound D, 0.73 g of Compound B, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 1.55 g (Yield 80%) of the target product as a white solid.

The chemical shift values of the target product measured by ¹H-NMR were 8.21 (d, 1H), 8.20 (d, 1H), 7.61-7.54 (m, 4H), 7.51-7.43 (m, 4H), 7.41-7.37 (m, 7H), 7.35-7.29 (m, 7H), 7.23-7.17 (m, 7H), 7.14-7.09 (m, 2H), 7.05-7.02 (m, 1H), 6.98-6.96 (m, 3H). The molecular weight of the target product as measured by FAB-MS was 715. Accordingly, the target product was clearly identified as Compound 16.

In one embodiment, the material for an organic EL device according to an embodiment of the present disclosure may be synthesized, for example, as follows:

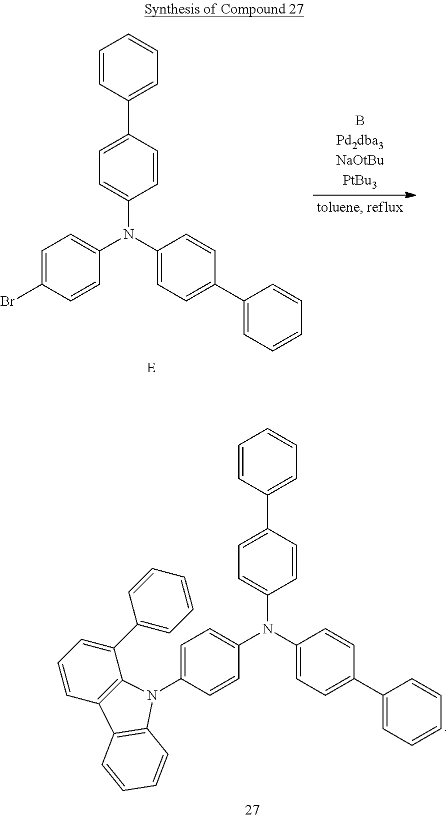

Under an argon atmosphere, 1.00 g of Compound E, 0.73 g of Compound B, 0.14 g of bis(dibenzylideneacetone)palladium(0) (Pd(dba)₂), 0.15 g of tri-tert-butylphosphine ((t-Bu)₃P), and 0.61 g of sodium tert-butoxide were added to a 100 mL, three-necked flask, followed by refluxing in 25 mL of toluene for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 1.10 g (Yield 82%) of a target product as a white solid.

The chemical shift values of the target product measured by ¹H-NMR were 8.23 (d, 1H), 8.21 (d, 1H), 7.94-7.90 (m, 4H), 7.56-7.49 (m, 7H), 7.40-7.38 (m, 7H), 7.35-7.29 (m, 7H), 7.15-7.11 (m, 2H), 6.99-6.95 (m, 4H). The molecular weight of the target product measured by FAB-MS was 639. Accordingly, the target product was clearly identified as Compound 27.

In one embodiment, the material for an organic EL device according to an embodiment of the present disclosure may be synthesized, for example, as follows:

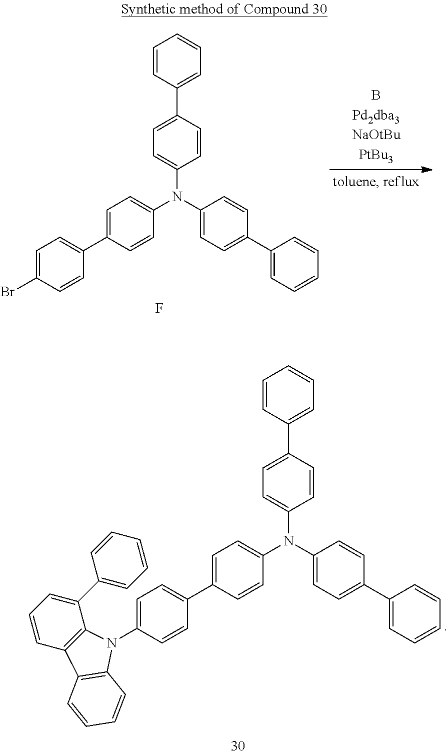

Under an argon atmosphere, 1.50 g of Compound F, 0.73 g of Compound B, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 1.77 g (Yield 91%) of a target product as a white solid.

The chemical shift values of the target product measured by ¹H-NMR were 8.22 (d, 1H), 8.20 (d, 1H), 7.94-7.91 (m, 4H), 7.66-7.60 (m, 7H), 7.58-7.53 (m, 7H), 7.49-7.36 (m, 7H), 7.15-7.11 (m, 2H), 7.05-7.03 (m, 1H), 6.98-6.65 (m, 3H). The molecular weight of the target product as measured by FAB-MS was 715. Accordingly, the target product was clearly identified as Compound 30.

In one embodiment, the material for an organic EL device according to an embodiment of the present disclosure may be synthesized, for example, as follows:

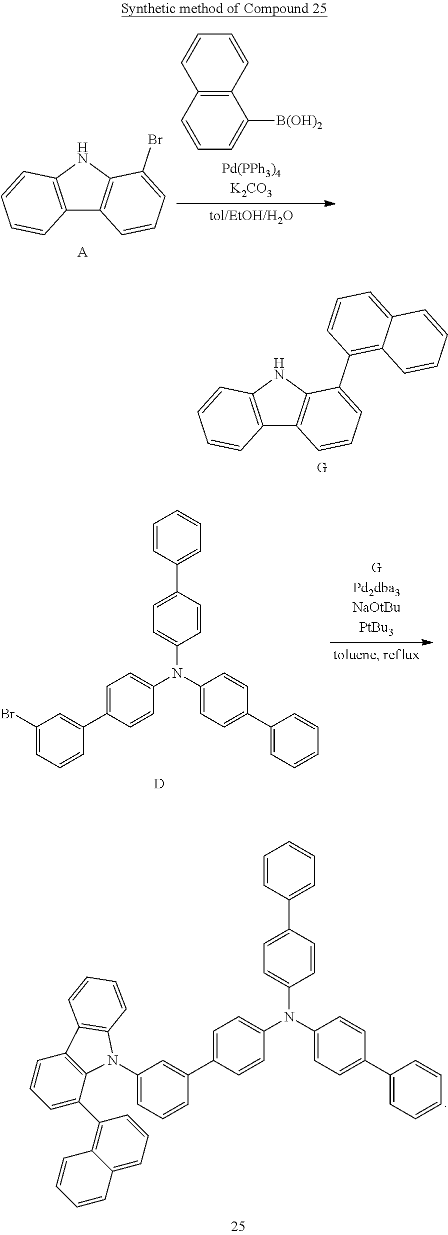

Under an argon atmosphere, 10.0 g of Compound A, 8.4 g of naphthylboronic acid, 3.28 g of tetrakistriphenylphosphine palladium(0) (Pd(PPh₃)₄), and 11.2 g of potassium carbonate were added to a 500 mL, three-necked flask, followed by heating in 200 mL of toluene, 20 mL of ethanol, and 20 mL of water at a temperature of 90° C. for 6 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 6.55 g (Yield 55%) of Compound G as a white solid.

Next, under an argon atmosphere, 1.50 g of Compound D, 0.88 g of Compound G, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 0.98 g (Yield 47%) of a target product as a white solid.

The chemical shift values of the target product measured by ¹H-NMR were 8.21 (d, 1H), 8.19 (d, 1H), 7.96-7.93 (m, 4H), 7.88-7.85 (m, 4H), 7.66-7.60 (m, 7H), 7.58-7.53 (m, 7H), 7.49-7.36 (m, 7H), 7.28-7.25 (m, 4H), 7.10-7.08 (m, 1H), 6.98-6.95 (m, 3H). The molecular weight of the target product as measured by FAB-MS was 765. Accordingly, the target product was clearly identified as Compound 25.

In one embodiment, the material for an organic EL device according to an embodiment of the present disclosure may be synthesized, for example, as follows:

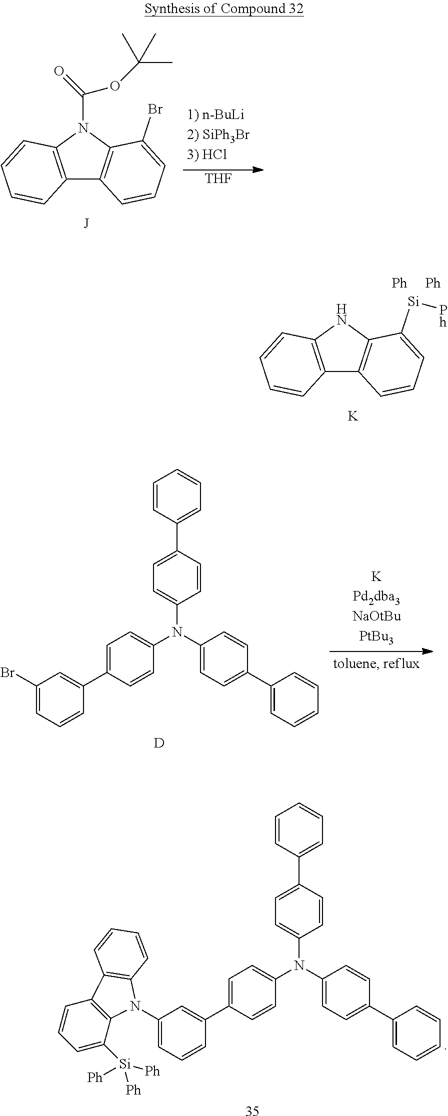

Under an argon atmosphere, 10.0 g of Compound H, 4.5 g of phenylboronic acid, 2.52 g of tetrakistriphenylphosphine palladium(0) (Pd(PPh₃)₄), and 8.58 g of potassium carbonate were added to a 500 mL, three-necked flask, followed by heating in 200 mL of toluene, 20 mL of ethanol, and 20 mL of water at a temperature of 90° C. for 6 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 6.30 g (Yield 64%) of Compound I as a white solid.

Next, under an argon atmosphere, 1.50 g of Compound D, 0.95 g of Compound I, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and the solvents were removed by distillation. The crude product thus obtained was separated by silica gel column chromatography (using a mixture of dichloromethane and hexane) and recrystallized using a mixture of toluene and hexane to obtain 1.69 g (Yield 79%) of a target product as a white solid.

The chemical shift values of the target product measured by ¹H-NMR were 8.29 (d, 1H), 8.28 (d, 1H), 8.88-8.86 (m, 3H), 7.98-7.97 (m, 4H), 7.66-7.60 (m, 7H), 7.58-7.50 (m, 12H), 7.45-7.36 (m, 7H), 7.19-7.15 (m, 2H), 7.06-7.04 (m, 1H), 6.96-6.92 (m, 3H). The molecular weight of the target product as measured by FAB-MS was 791. Accordingly, the target product was clearly identified as Compound 32.

In one embodiment, the material for an organic EL device according to an embodiment of the present disclosure may be synthesized, for example, as follows:

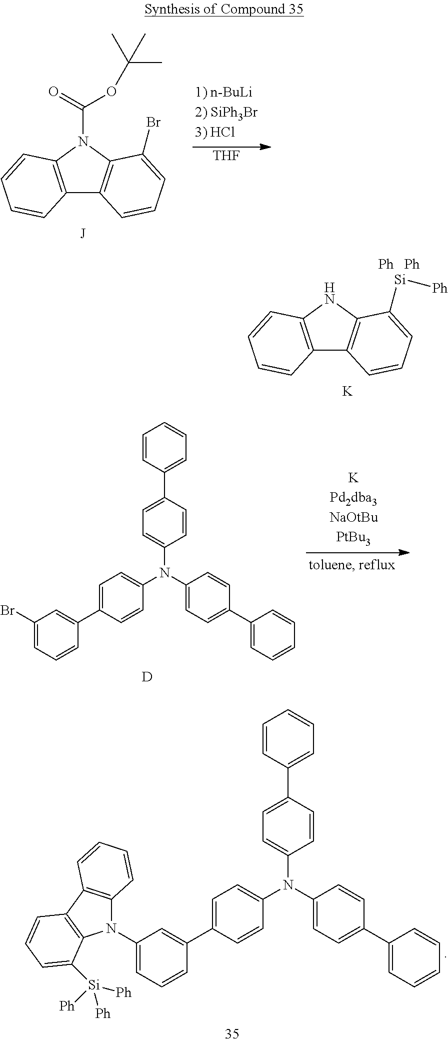

Under an argon atmosphere, to a 500 mL, three-necked flask, 10.0 g of Compound J was added and dissolved in 150 mL of THF, and 18.1 mL of n-BuLi was added thereto at −78° C. After 1 hour, 11.8 g of triphenylsilylbromide (SiPh₃Br) was added thereto, followed by stirring at room temperature for 8 hours. After that, 20 mL of 1 N aqueous hydrochloric acid (HCl) was added and stirred for 5 minutes, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 8.34 g (Yield 62%) of Compound K as a white solid.

Under an argon atmosphere, 1.50 g of Compound D, 0.95 g of Compound K, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and solvents were distilled. The crude product thus obtained was separated by silica gel column chromatography (using a mixture of dichloromethane and hexane) and recrystallized using a mixture of toluene and hexane to obtain 1.69 g (Yield 79%) of a target product as a white solid.

The chemical shift values of the target product measured by ¹H-NMR were 8.22 (d, 1H), 8.19 (d, 1H), 7.63-7.60 (m, 4H), 7.47.43 (m, 4H), 7.41-7.37 (m, 7H), 7.34-7.28 (m, 12), 7.26-7.17 (m, 12H), 7.15-7.11 (m, 2H), 7.04-7.01 (m, 1H), 6.98-6.96 (m, 3H). The molecular weight of the target product as measured by FAB-MS was 897. Accordingly, the target product was clearly identified as Compound 35.

In some embodiments, the material for an organic EL device according to an embodiment of the present disclosure may be synthesized, for example, as follows:

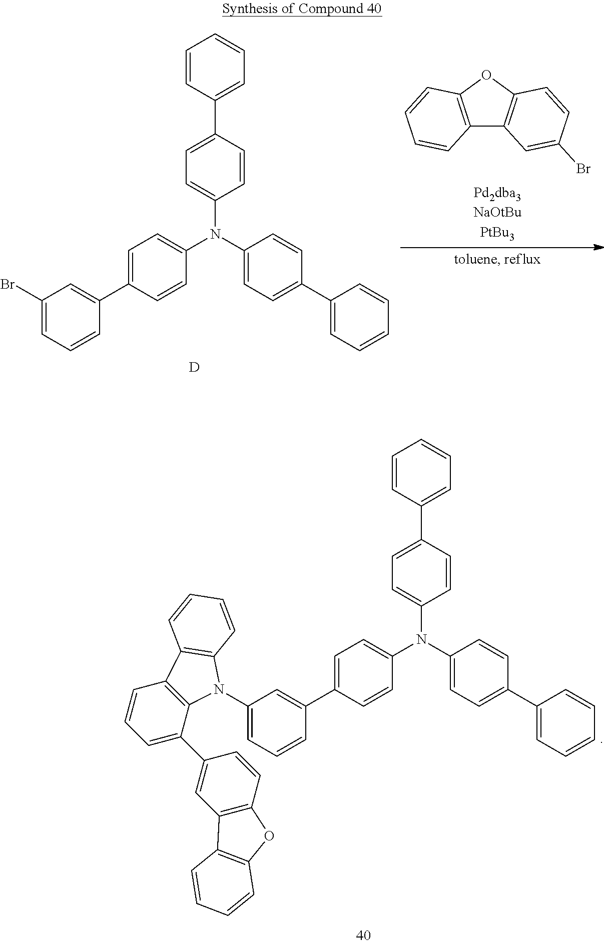

Under an argon atmosphere, 1.50 g of Compound D, 0.95 g of 3-bromodibenzofuran, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and solvents were removed by distillation. The crude product thus obtained was separated by silica gel column chromatography (using a mixture of dichloromethane and hexane) and recrystallized using a mixture of toluene and hexane to obtain 1.69 g (Yield 79%) of a target product as a white solid.

The chemical shift values of the target product measured by ¹H-NMR were 8.21 (d, 1H), 8.20 (d, 1H), 7.59-7.55 (m, 4H), 7.50-7.43 (m, 4H), 7.39-7.34 (m, 8H), 7.32-7.26 (m, 7H), 7.23-7.18 (m, 7H), 7.10-7.06 (m, 2H), 7.03-6.99 (m, 1H), 6.95-6.92 (m, 3H). The molecular weight of the target product measured by FAB-MS was 805. Accordingly, the target product was clearly identified as Compound 40.

In one embodiment, the material for an organic EL device according to an embodiment of the present disclosure may be synthesized, for example, as follows:

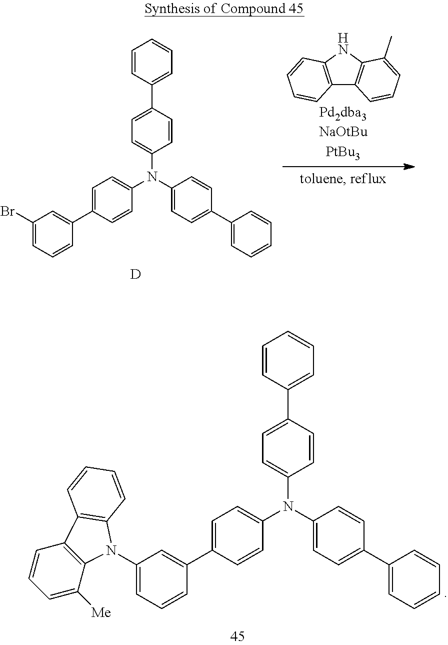

Under an argon atmosphere, 1.50 g of Compound D, 0.95 g of 1-methylcarbazole, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and the solvents were removed by distillation. The crude product thus obtained was separated by silica gel column chromatography (using a mixture of dichloromethane and hexane) and recrystallized using a mixture of toluene and hexane to obtain 1.69 g (Yield 79%) of a target product as a white solid.

The chemical shift values of the target product measured by ¹H-NMR were 8.18 (d, 1H), 8.16 (d, 1H), 7.59-7.53 (m, 4H), 7.49-7.44 (m, 4H), 7.39-7.36 (m, 5H), 7.32-7.25 (m, 6H), 7.19-7.15 (m, 5H), 7.08-7.06 (m, 2H), 7.04-7.01 (m, 1H), 6.98-6.96 (m, 3H), 1.92 (3H, s). The molecular weight of the target product as measured by FAB-MS was 652. Accordingly, the target product was clearly identified as Compound 45.

In one embodiment, the material for an organic EL device according to an embodiment of the present disclosure may be synthesized, for example, as follows:

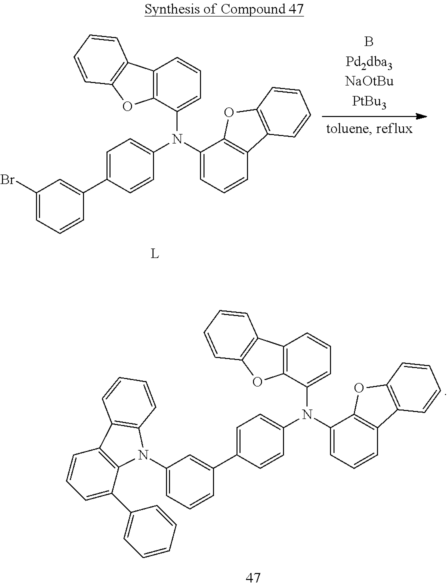 [SMILES: Brc1cccc(-c2ccc(N(c3cccc4c3oc3ccccc34)c3cccc4c3oc3ccccc34)cc2)c1 ; c1ccc(-c2cccc3c4ccccc4n(-c4cccc(-c5ccc(N(c6cccc7c6oc6ccccc67)c6cccc7c6oc6ccccc67)cc5)c4)c23)cc1]

Under an argon atmosphere, 1.50 g of Compound L, 0.95 g of Compound B, 0.17 g of bis(dibenzylideneacetone)palladium(0) (Pd(dba)₂), 0.15 g of tri-tert-butylphosphine ((t-Bu)₃P), and 0.78 g of sodium tert-butoxide were added to a 100 mL, three-necked flask, followed by refluxing in 25 mL of toluene for 3 hours. After cooling in the air, water was added, an organic layer was separated, and the solvents were removed by distillation. The crude product thus obtained was separated by silica gel column chromatography (using a mixture of dichloromethane and hexane) and recrystallized using a mixture of toluene and hexane to obtain 1.69 g (Yield 79%) of a target product as a white solid.

The chemical shift values of the target product measured by ¹H-NMR were 8.21 (d, 1H), 8.20 (d, 1H), 7.61-7.54 (m, 4H), 7.51-7.43 (m, 4H), 7.41-7.37 (m, 5H), 7.35-7.29 (m, 7H), 7.23-7.17 (m, 5H), 7.14-7.09 (m, 2H), 7.05-7.02 (m, 1H), 6.98-6.96 (m, 3H). The molecular weight of the target product as measured by FAB-MS was 743. Accordingly, the target product was clearly identified as Compound 47.

Organic EL devices according to Examples 1 to 10 were manufactured using Compounds 1, 16, 27, 30, 25, 32, 35, 40, 45, and 47, respectively, as hole transport materials:

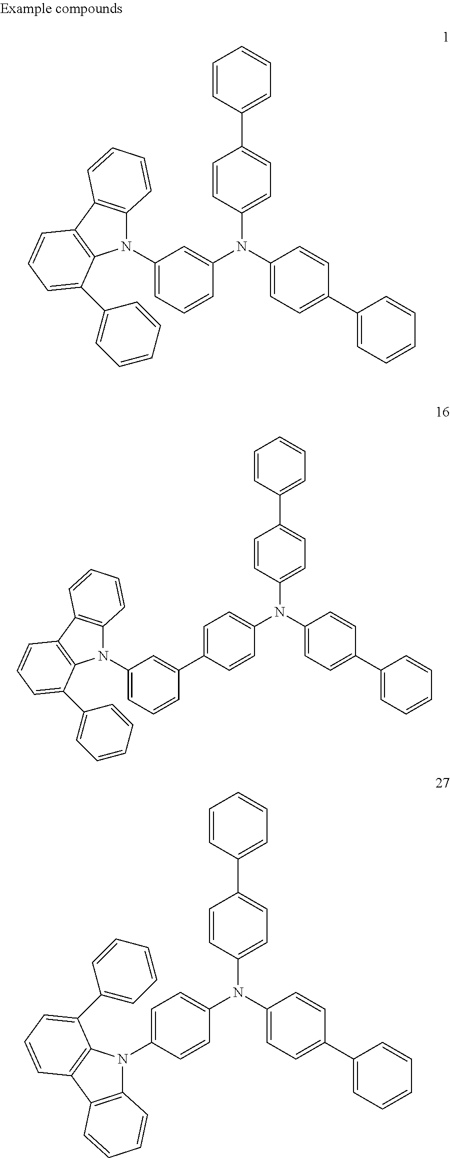 [SMILES: c1ccc(-c2ccc(N(c3ccc(-c4ccccc4)cc3)c3cccc(-n4c5ccccc5c5cccc(-c6ccccc6)c54)c3)cc2)cc1 ; c1ccc(-c2ccc(N(c3ccc(-c4ccccc4)cc3)c3ccc(-c4cccc(-n5c6ccccc6c6cccc(-c7ccccc7)c65)c4)cc3)cc2)cc1 ; c1ccc(-c2ccc(N(c3ccc(-c4ccccc4)cc3)c3ccc(-n4c5ccccc5c5cccc(-c6ccccc6)c54)cc3)cc2)cc1]

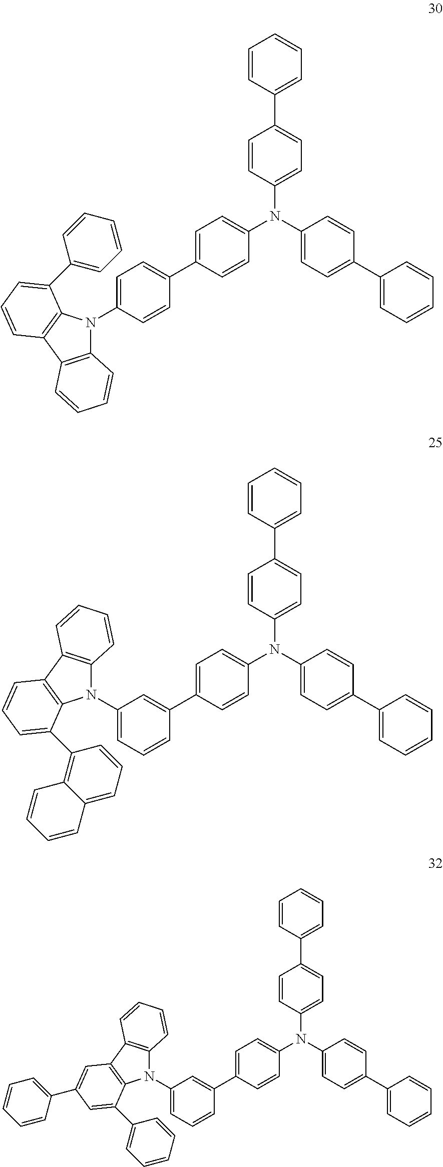 [SMILES: c1ccc(-c2ccc(N(c3ccc(-c4ccccc4)cc3)c3ccc(-c4ccc(-n5c6ccccc6c6cccc(-c7ccccc7)c65)cc4)cc3)cc2)cc1 ; c1ccc(-c2ccc(N(c3ccc(-c4ccccc4)cc3)c3ccc(-c4cccc(-n5c6ccccc6c6cccc(-c7cccc8ccccc78)c65)c4)cc3)cc2)cc1 ; c1ccc(-c2ccc(N(c3ccc(-c4ccccc4)cc3)c3ccc(-c4cccc(-n5c6ccccc6c6cc(-c7ccccc7)cc(-c7ccccc7)c65)c4)cc3)cc2)cc1]

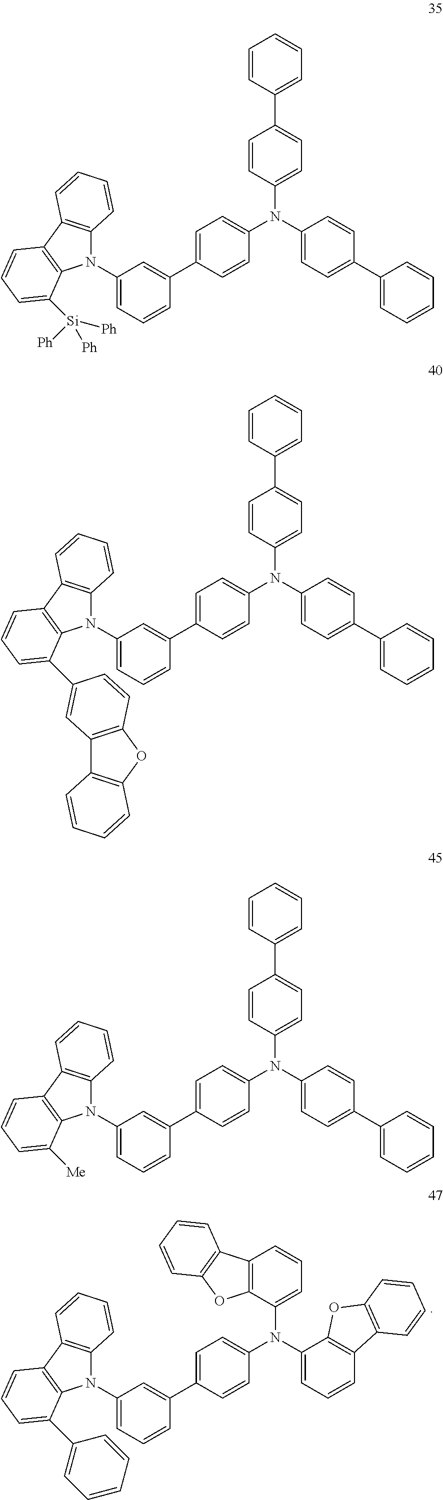 [SMILES: c1ccc(-c2ccc(N(c3ccc(-c4ccccc4)cc3)c3ccc(-c4cccc(-n5c6ccccc6c6cccc([Si](c7ccccc7)(c7ccccc7)c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8oc9ccccc9c8c7)c65)c4)cc3)cc2)cc1 ; Cc1cccc2c3ccccc3n(-c3cccc(-c4ccc(N(c5ccc(-c6ccccc6)cc5)c5ccc(-c6ccccc6)cc5)cc4)c3)c12 ; c1ccc(-c2cccc3c4ccccc4n(-c4cccc(-c5ccc(N(c6cccc7c6oc6ccccc67)c6cccc7c6oc6ccccc67)cc5)c4)c23)cc1]

For comparison, organic EL devices according to Comparative Examples 1 to 4 were manufactured using Comparative Compounds C1 to C4, respectively, as hole transport materials:

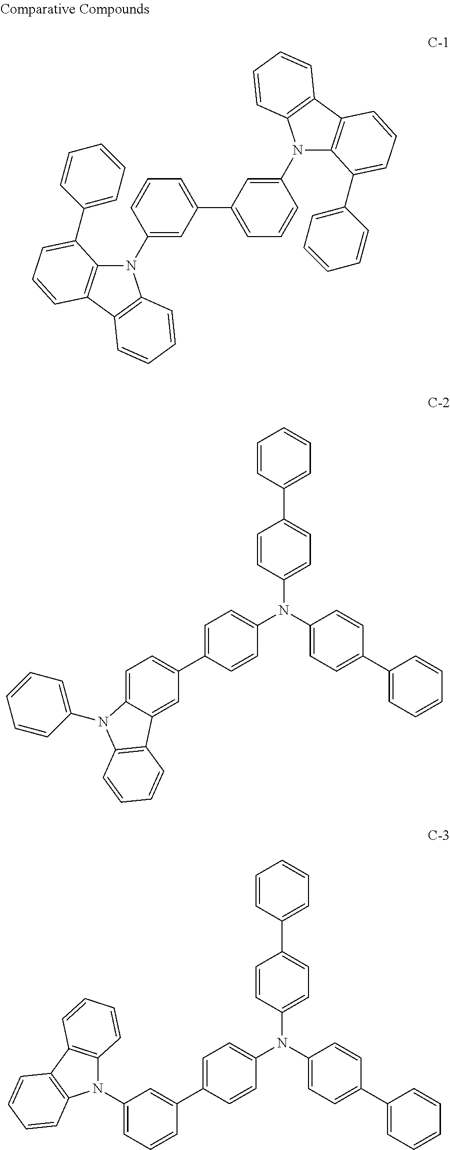 [SMILES: c1ccc(-c2cccc3c4ccccc4n(-c4cccc(-c5cccc(-n6c7ccccc7c7cccc(-c8ccccc8)c76)c5)c4)c23)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(-c2ccc(N(c3ccc(-c4ccccc4)cc3)c3ccc(-c4cccc(-n5c6ccccc6c6ccccc65)c4)cc3)cc2)cc1]

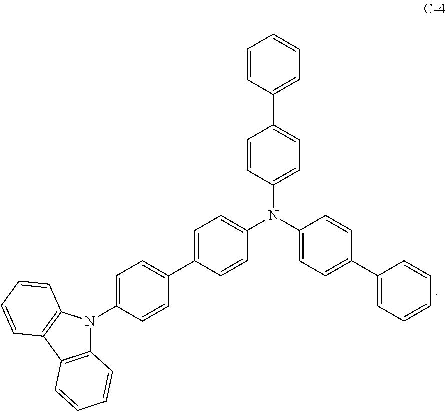 [SMILES: c1ccc(-c2ccc(N(c3ccc(-c4ccccc4)cc3)c3ccc(-c4ccc(-n5c6ccccc6c6ccccc65)cc4)cc3)cc2)cc1]

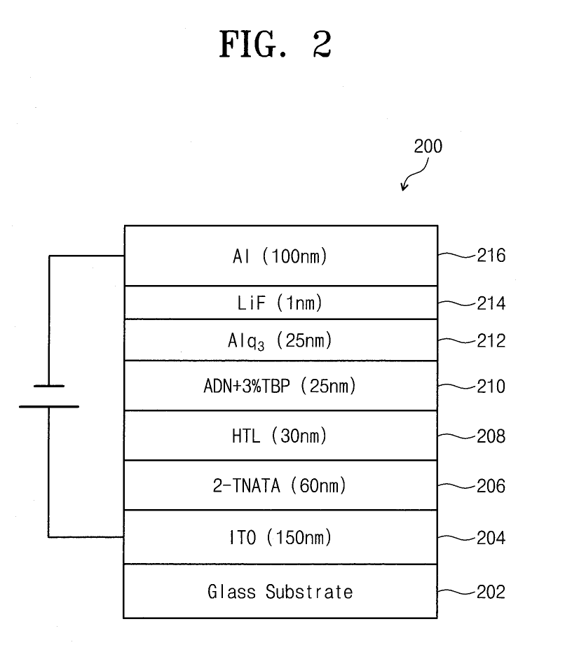

An organic EL device 200 according to an embodiment of the present disclosure is illustrated in FIG. 2. In this embodiment, a transparent glass substrate was used as a substrate 202, an anode 204 was formed using ITO to a layer thickness of about 150 nm, a hole injection layer 206 was formed using 2-TNATA to a layer thickness of about 60 nm, a hole transport layer 208 was formed using a hole transport layer material to a layer thickness of about 30 nm, an emission layer 210 was formed using ADN doped with 3% TBP to a layer thickness of about 25 nm, an electron transport layer 212 was formed using Alq₃ to a layer thickness of about 25 nm, an electron injection layer 214 was formed using LiF to a layer thickness of about 1 nm, and a cathode 216 was formed using Al to a layer thickness of about 100 nm. Each layer of the organic EL device 200 was formed using a vacuum deposition method.

The organic EL devices according to Comparative Examples 1 to 4 were manufactured according to substantially the same method used to manufacture the above organic EL device 200, except for using Compounds C-1 to C-4 as the material for each hole transport layer.

The emission efficiency of each organic EL device 200 thus manufactured was evaluated. A C9920-11 brightness light distribution characteristics measurement system by HAMAMATSU Photonics Co. was used to evaluate the emission efficiency at a current density of 10 mA/cm². The evaluation results are shown in Table 1:

TABLE 1

| Device manufacturing | | Emission efficiency |
|---|---|---|
| example | Hole transport layer | (cd/A) |
| Example 1 | Compound 1 | 7.9 |
| Example 2 | Compound 16 | 8.5 |
| Example 3 | Compound 27 | 7.0 |
| Example 4 | Compound 30 | 7.9 |
| Example 5 | Compound 25 | 8.0 |
| Example 6 | Compound 32 | 8.5 |
| Example 7 | Compound 35 | 7.8 |
| Example 8 | Compound 40 | 7.8 |
| Example 9 | Compound 45 | 6.8 |
| Example 10 | Compound 47 | 8.0 |
| Comparative | Comparative | 4 |
| Example 1 | Compound C-1 | |
| Comparative | Comparative | 5.2 |
| Example 2 | Compound C-2 | |
| Comparative | Comparative | 6.4 |
| Example 3 | Compound C-3 | |
| Comparative | Comparative | 6.0 |
| Example 4 | Compound C-4 | |

Referring to the results in Table 1, the efficiency was found to be improved in each of the organic EL devices according to Examples 1 to 10, compared to those according to Comparative Examples 1 to 4. In Comparative Example 1 using Compound C-1 (composed of only a carbazole group) as the hole transport material, the emission efficiency was markedly lower than in Examples 1 to 10. These results may be due to the lower hole transport capacity of Compound C-1 compared to a tertiary amine compound. Examples 1 to 10, which include the amine compounds of the present disclosure (in which position 1 of a carbazolyl group is substituted with a phenyl group, a naphthyl group, a silyl group, a methyl group, or a dibenzofuranyl group) exhibited higher efficiencies than Comparative Compounds C-1 to C-4. In the example compounds, since position 9 (e.g., the N atom) of the carbazolyl group is combined (e.g., coupled) via L (linker) with a nitrogen atom of an amine group, and position 1 of the carbazolyl group is substituted with a phenyl group, a naphthyl group, or a silyl group, the whole molecule of the amine compound of the present disclosure may be distorted (e.g., the molecule as a whole may be non-planar), the volume of the molecule may increase, and the HOMO-LUMO energy gap may increase. It is thought that this may improve or increase the emission efficiency of the organic EL device. However, the present disclosure is not limited to any particular mechanism or theory.

Example 9 includes an amine compound having a carbazolyl group substituted with a methyl group, and has the lowest efficiency among the organic EL devices of Examples 1 to 10. Since the methyl group has a relatively small volume, distortion due to steric hindrance may be decreased relative to compounds including larger substituents. When comparing Example 1 with Example 3, and Example 2 with Example 4, respectively, higher emission efficiencies were obtained when L (linker) in the amine compound represented by Formula 1 was an m-biphenylene group compared to an m-phenylene group.

From the results in Table 1, the organic EL devices using the material for an organic EL device according to an embodiment of the present disclosure as hole transport materials were recognized to show higher efficiency when compared to organic EL devices using the comparative compounds as hole transport materials. The material for an organic EL device according to an embodiment of the present disclosure may enable high device efficiency when position 9 (e.g., the N atom) of a carbazolyl group is combined (e.g., coupled) via L (linker) with the nitrogen atom of an amine group, and at least one selected from positions 1 and 8 of the carbazolyl group is substituted with an aryl group, a heteroaryl group, an alkyl group, or a silyl group.

According to the present disclosure, a material for an organic EL device attaining high emission efficiency and an organic EL device using the same are provided. In the material for an organic EL device according to the present disclosure, position 9 (e.g., the N atom) in a carbazolyl group may be combined (e.g., coupled) via L (linker) with a nitrogen atom of an amine group, and at least one selected from positions 1 and 8 of the carbazolyl group may be substituted with an aryl group, a heteroaryl group, an alkyl group, or a silyl group. Accordingly, the whole molecule of the compound for an organic EL device may be distorted (e.g., the molecule as a whole may be non-planar), the volume of the molecule may increase, and the HOMO-LUMO energy gap may increase. Therefore, the emission efficiency of the organic EL device may be improved.

As used herein, expressions such as “at least one of”, “one of”, and “selected from”, when preceding a list of elements, modify the entire list of elements and do not modify the individual elements of the list. Further, the use of “may” when describing embodiments of the present disclosure refers to “one or more embodiments of the present disclosure”.

In addition, as used herein, the terms “use”, “using”, and “used” may be considered synonymous with the terms “utilize”, “utilizing”, and “utilized”, respectively.

As used herein, the terms “substantially”, “about”, and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Although example embodiments of the present disclosure have been described, it is understood that the present disclosure should not be limited to these example embodiments, and that various changes and modifications can be made by one of ordinary skill in the art within the spirit and scope of the present disclosure, as defined by the following claims and equivalents thereof.

What is claimed is:

1. A material for an organic electroluminescent (EL) device, represented by Formula 1: 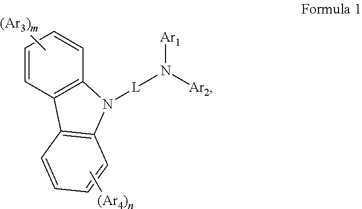
wherein in Formula 1,
Ar₁ and Ar₂ are each independently a substituted or unsubstituted aryl group having 6 to 30 carbon atoms for forming a ring, or a substituted or unsubstituted heteroaryl group having 1 to 30 carbon atoms for forming a ring,
Ar₃ and Ar₄ are each independently a substituted or unsubstituted aryl group having 6 to 30 carbon atoms for forming a ring, a substituted or unsubstituted heteroaryl group having 5 to 30 carbon atoms for forming a ring, an alkyl group having 1 to 6 carbon atoms, or a silyl group,
L is a substituted or unsubstituted arylene group having 6 to 30 carbon atoms for forming a ring, or a substituted or unsubstituted heteroarylene group having 5 to 30 carbon atoms for forming a ring,
m and n are each independently an integer selected from 0 to 4,
m+n≧1, and
at least one of Ar₃ or Ar₄ is substituted in at least one of position 1 or position 8 of a carbazolyl group.

2. The material for an organic EL device of claim 1, wherein in Formula 1, m+n=1.

3. The material for an organic EL device of claim 1, wherein Ar₃ and/or Ar₄ are each independently a phenyl group, a naphthyl group, a biphenyl group, a fluoroaryl group, a dibenzofuranyl group, or a silyl group.

4. The material for an organic EL device of claim 1, wherein L is selected from arylene groups (L-1) to (L-7):

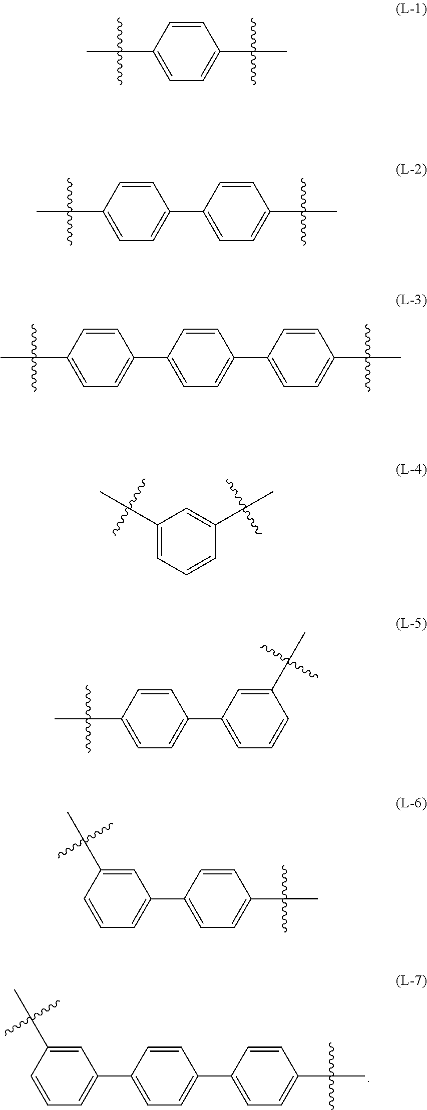

5. The material for an organic EL device of claim 1, wherein L is an m-phenylene group.

6. The material for an organic EL device of claim 1, wherein the material for an organic EL device represented by Formula 1 is represented by at least one selected from Compounds 1 to 50:

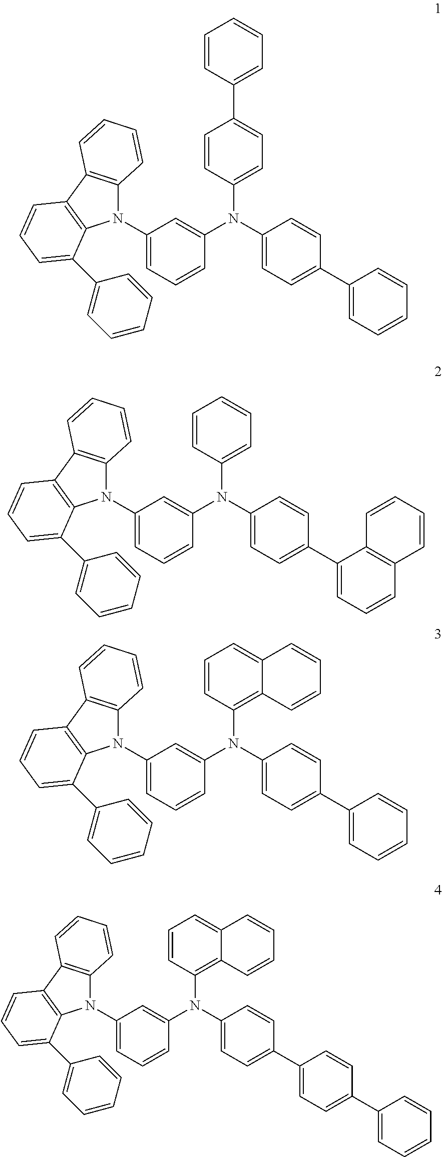

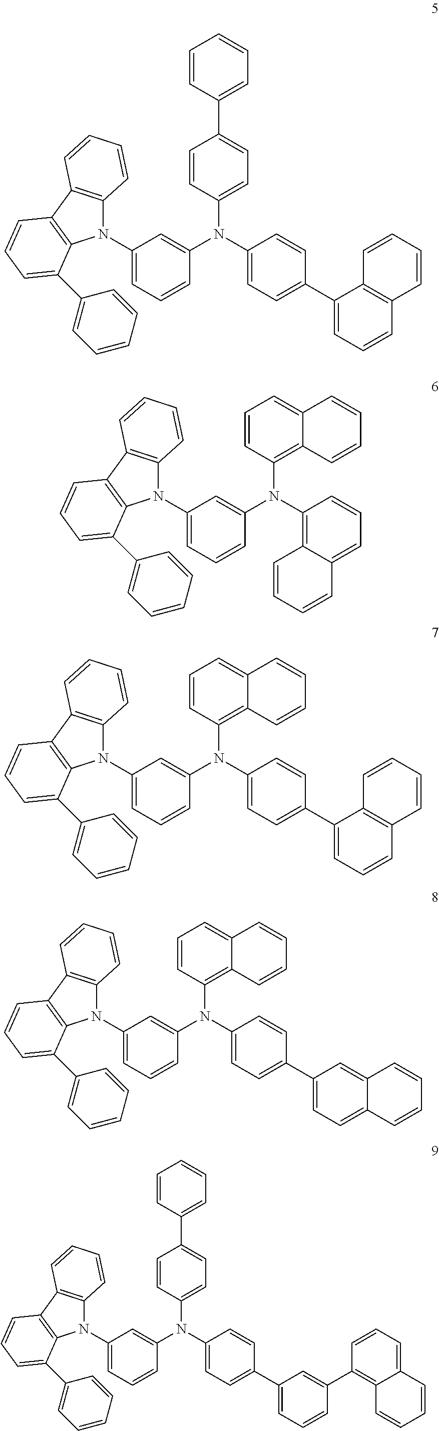 [SMILES: c1ccc(-c2ccc(N(c3ccc(-c4cccc5ccccc45)cc3)c3cccc(-n4c5ccccc5c5cccc(-c6ccccc6)c54)c3)cc2)cc1 ; c1ccc(-c2cccc3c4ccccc4n(-c4cccc(N(c5cccc6ccccc56)c5cccc6ccccc56)c4)c23)cc1 ; c1ccc(-c2cccc3c4ccccc4n(-c4cccc(N(c5ccc(-c6cccc7ccccc67)cc5)c5cccc6ccccc56)c4)c23)cc1 ; c1ccc(-c2cccc3c4ccccc4n(-c4cccc(N(c5ccc(-c6ccc7ccccc7c6)cc5)c5cccc6ccccc56)c4)c23)cc1 ; c1ccc(-c2ccc(N(c3ccc(-c4cccc(-c5cccc6ccccc56)c4)cc3)c3cccc(-n4c5ccccc5c5cccc(-c6ccccc6)c54)c3)cc2)cc1]

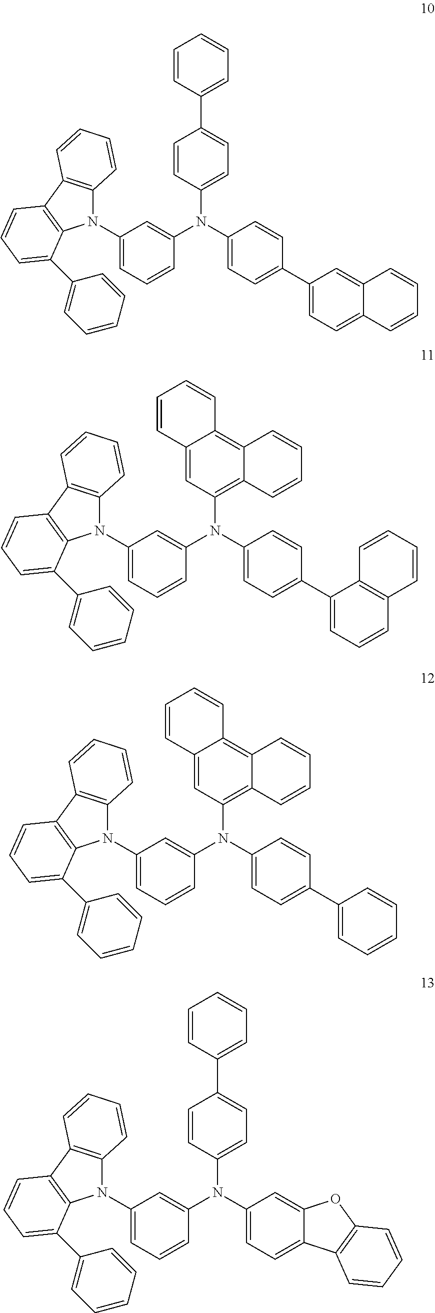 [SMILES: c1ccc(-c2ccc(N(c3ccc(-c4ccc5ccccc5c4)cc3)c3cccc(-n4c5ccccc5c5cccc(-c6ccccc6)c54)c3)cc2)cc1 ; c1ccc(-c2cccc3c4ccccc4n(-c4cccc(N(c5ccc(-c6cccc7ccccc67)cc5)c5cc6ccccc6c6ccccc56)c4)c23)cc1 ; c1ccc(-c2ccc(N(c3cccc(-n4c5ccccc5c5cccc(-c6ccccc6)c54)c3)c3cc4ccccc4c4ccccc34)cc2)cc1 ; c1ccc(-c2ccc(N(c3cccc(-n4c5ccccc5c5cccc(-c6ccccc6)c54)c3)c3ccc4c(c3)oc3ccccc34)cc2)cc1]

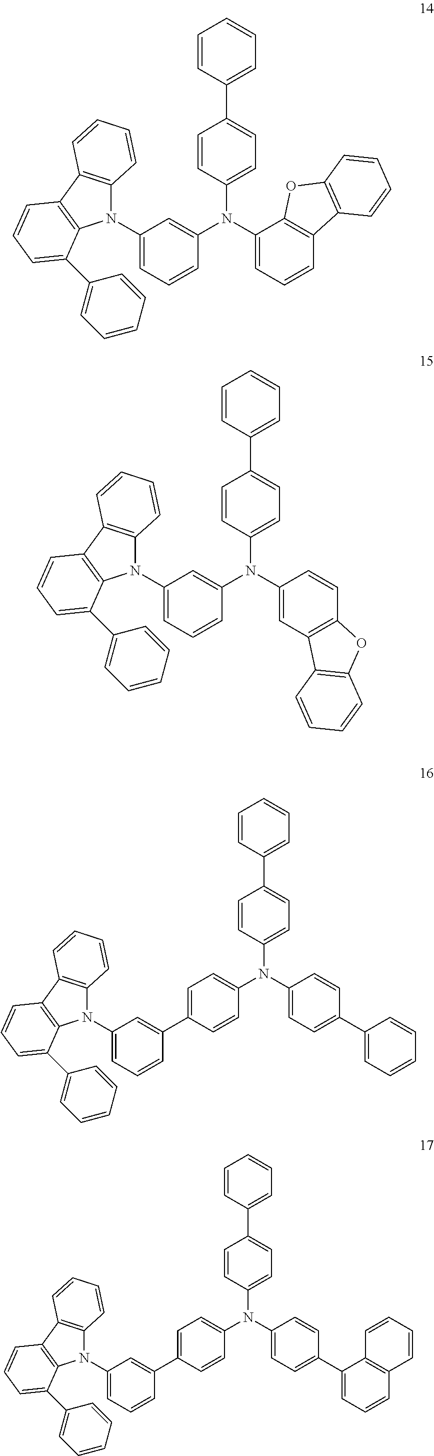 [SMILES: c1ccc(-c2ccc(N(c3cccc(-n4c5ccccc5c5cccc(-c6ccccc6)c54)c3)c3cccc4c3oc3ccccc34)cc2)cc1 ; c1ccc(-c2ccc(N(c3cccc(-n4c5ccccc5c5cccc(-c6ccccc6)c54)c3)c3ccc4oc5ccccc5c4c3)cc2)cc1 ; c1ccc(-c2ccc(N(c3ccc(-c4ccccc4)cc3)c3ccc(-c4cccc(-n5c6ccccc6c6cccc(-c7ccccc7)c65)c4)cc3)cc2)cc1 ; c1ccc(-c2ccc(N(c3ccc(-c4cccc(-n5c6ccccc6c6cccc(-c7ccccc7)c65)c4)cc3)c3ccc(-c4cccc5ccccc45)cc3)cc2)cc1]

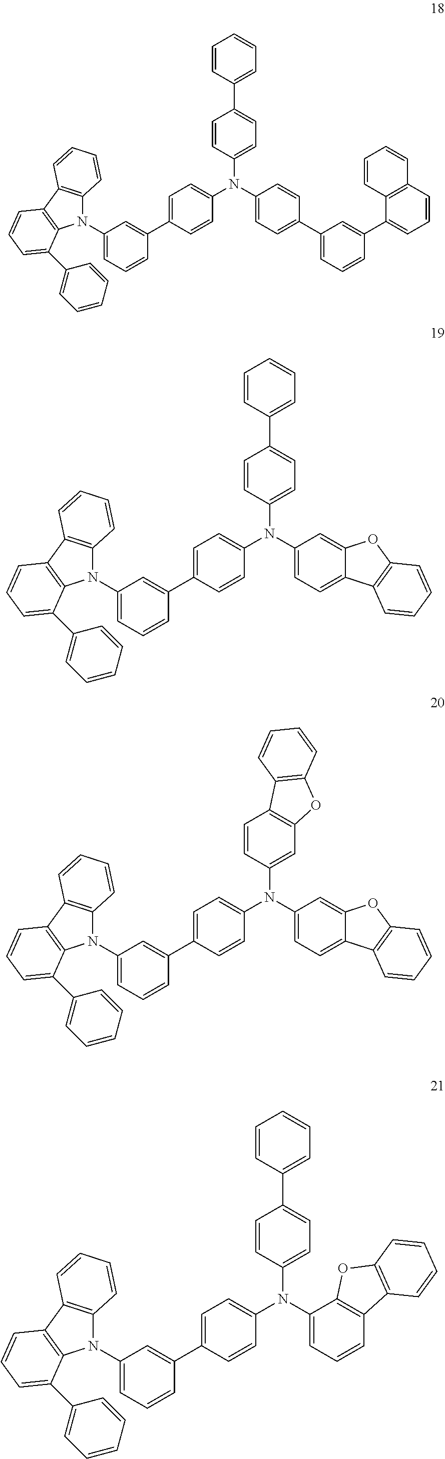 [SMILES: c1ccc(-c2ccc(N(c3ccc(-c4cccc(-c5cccc6ccccc56)c4)cc3)c3ccc(-c4cccc(-n5c6ccccc6c6cccc(-c7ccccc7)c65)c4)cc3)cc2)cc1 ; c1ccc(-c2ccc(N(c3ccc(-c4cccc(-n5c6ccccc6c6cccc(-c7ccccc7)c65)c4)cc3)c3ccc4c(c3)oc3ccccc34)cc2)cc1 ; c1ccc(-c2cccc3c4ccccc4n(-c4cccc(-c5ccc(N(c6ccc7c(c6)oc6ccccc67)c6ccc7c(c6)oc6ccccc67)cc5)c4)c23)cc1 ; c1ccc(-c2ccc(N(c3ccc(-c4cccc(-n5c6ccccc6c6cccc(-c7ccccc7)c65)c4)cc3)c3cccc4c3oc3ccccc34)cc2)cc1]

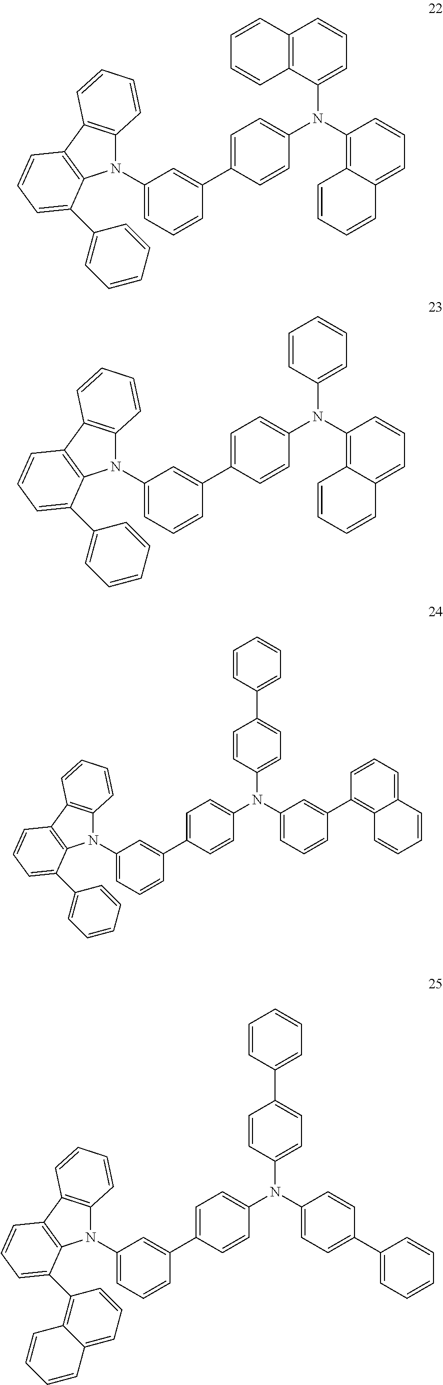 [SMILES: c1ccc(-c2cccc3c4ccccc4n(-c4cccc(-c5ccc(N(c6cccc7ccccc67)c6cccc7ccccc67)cc5)c4)c23)cc1 ; c1ccc(-c2cccc3c4ccccc4n(-c4cccc(-c5ccc(N(c6ccccc6)c6cccc7ccccc67)cc5)c4)c23)cc1 ; c1ccc(-c2ccc(N(c3ccc(-c4cccc(-n5c6ccccc6c6cccc(-c7ccccc7)c65)c4)cc3)c3cccc(-c4cccc5ccccc45)c3)cc2)cc1 ; c1ccc(-c2ccc(N(c3ccc(-c4ccccc4)cc3)c3ccc(-c4cccc(-n5c6ccccc6c6cccc(-c7cccc8ccccc78)c65)c4)cc3)cc2)cc1]

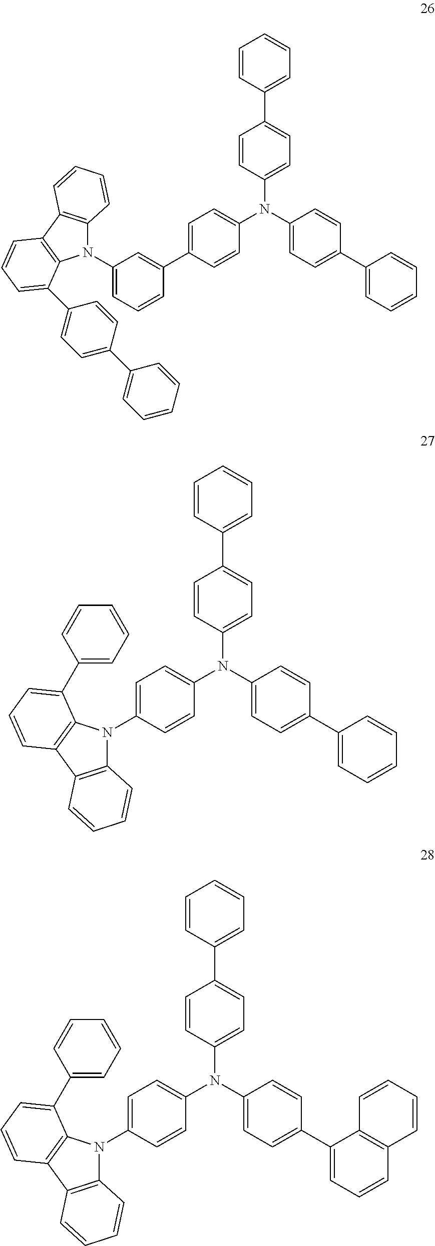 [SMILES: c1ccc(-c2ccc(-c3cccc4c5ccccc5n(-c5cccc(-c6ccc(N(c7ccc(-c8ccccc8)cc7)c7ccc(-c8ccccc8)cc7)cc6)c5)c34)cc2)cc1 ; c1ccc(-c2ccc(N(c3ccc(-c4ccccc4)cc3)c3ccc(-n4c5ccccc5c5cccc(-c6ccccc6)c54)cc3)cc2)cc1 ; c1ccc(-c2ccc(N(c3ccc(-c4cccc5ccccc45)cc3)c3ccc(-n4c5ccccc5c5cccc(-c6ccccc6)c54)cc3)cc2)cc1]

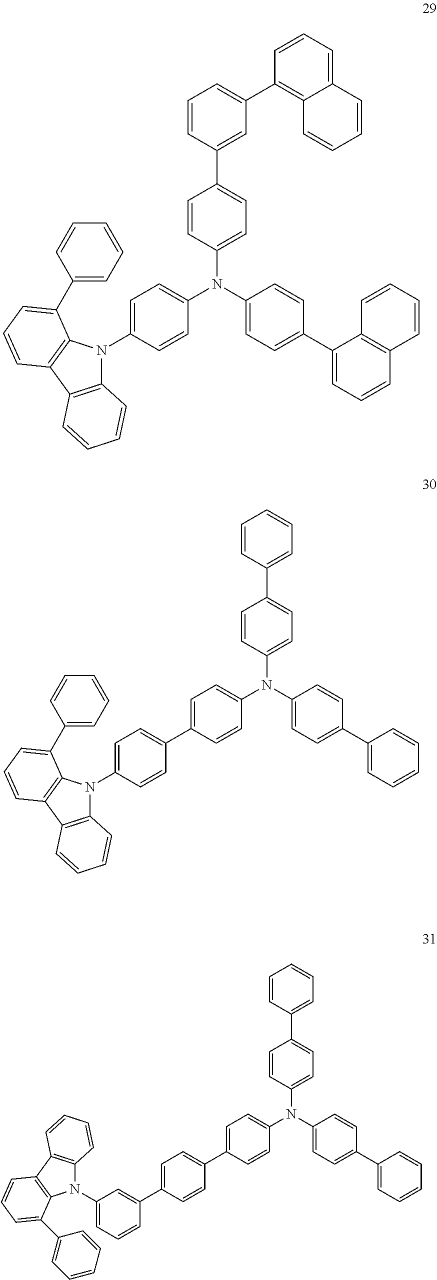 [SMILES: c1ccc(-c2cccc3c4ccccc4n(-c4ccc(N(c5ccc(-c6cccc(-c7cccc8ccccc78)c6)cc5)c5ccc(-c6cccc7ccccc67)cc5)cc4)c23)cc1 ; c1ccc(-c2ccc(N(c3ccc(-c4ccccc4)cc3)c3ccc(-c4ccc(-n5c6ccccc6c6cccc(-c7ccccc7)c65)cc4)cc3)cc2)cc1 ; c1ccc(-c2ccc(N(c3ccc(-c4ccccc4)cc3)c3ccc(-c4ccc(-c5cccc(-n6c7ccccc7c7cccc(-c8ccccc8)c76)c5)cc4)cc3)cc2)cc1]

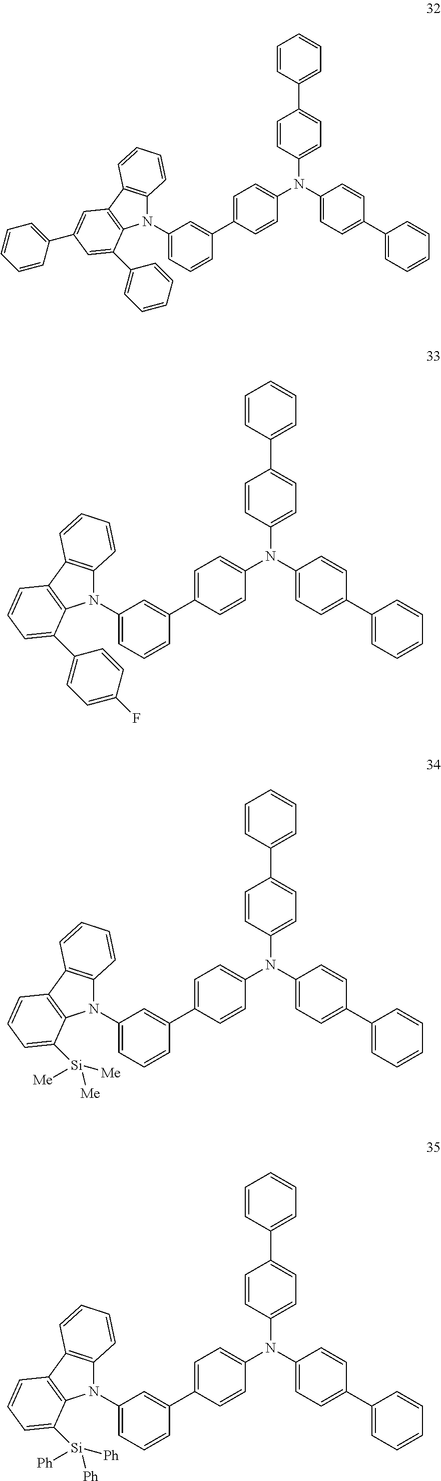 [SMILES: c1ccc(-c2ccc(N(c3ccc(-c4ccccc4)cc3)c3ccc(-c4cccc(-n5c6ccccc6c6cc(-c7ccccc7)cc(-c7ccccc7)c65)c4)cc3)cc2)cc1 ; Fc1ccc(-c2cccc3c4ccccc4n(-c4cccc(-c5ccc(N(c6ccc(-c7ccccc7)cc6)c6ccc(-c7ccccc7)cc6)cc5)c4)c23)cc1 ; C[Si](C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2ccc(N(c3ccc(-c4ccccc4)cc3)c3ccc(-c4cccc(-n5c6ccccc6c6cccc([Si](c7ccccc7)(c7ccccc7)c7ccccc7)c65)c4)cc3)cc2)cc1]

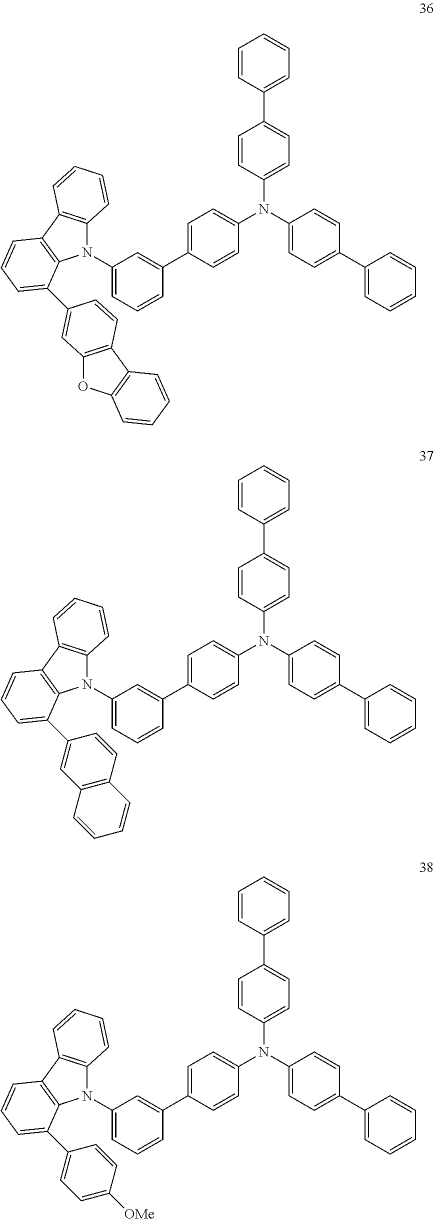 [SMILES: c1ccc(-c2ccc(N(c3ccc(-c4ccccc4)cc3)c3ccc(-c4cccc(-n5c6ccccc6c6cccc(-c7ccc8c(c7)oc7ccccc78)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8ccccc8c7)c65)c4)cc3)cc2)cc1 ; COc1ccc(-c2cccc3c4ccccc4n(-c4cccc(-c5ccc(N(c6ccc(-c7ccccc7)cc6)c6ccc(-c7ccccc7)cc6)cc5)c4)c23)cc1]

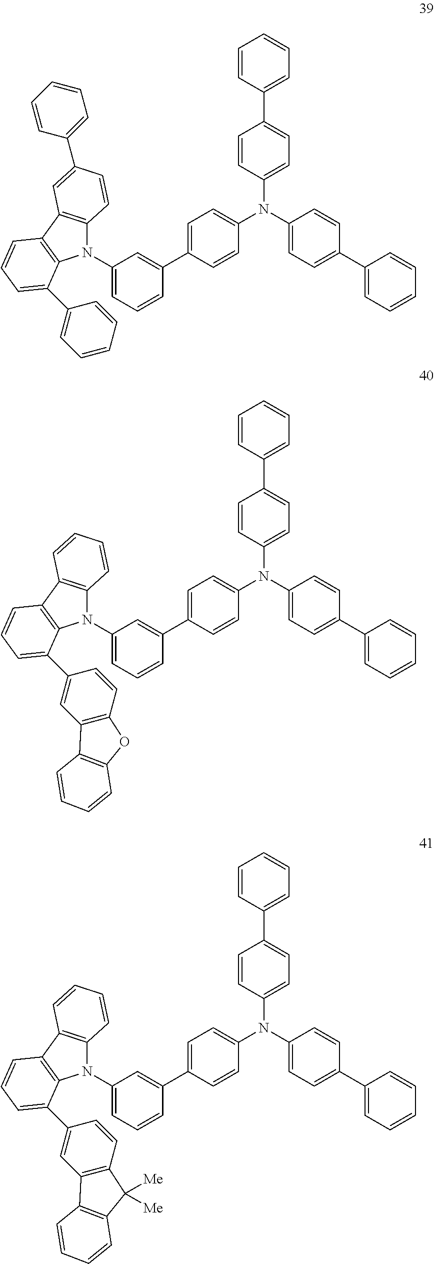 [SMILES: c1ccc(-c2ccc(N(c3ccc(-c4ccccc4)cc3)c3ccc(-c4cccc(-n5c6ccc(-c7ccccc7)cc6c6cccc(-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8oc9ccccc9c8c7)c65)c4)cc3)cc2)cc1 ; CC1(C)c2ccccc2-c2cc(-c3cccc4c5ccccc5n(-c5cccc(-c6ccc(N(c7ccc(-c8ccccc8)cc7)c7ccc(-c8ccccc8)cc7)cc6)c5)c34)ccc21]

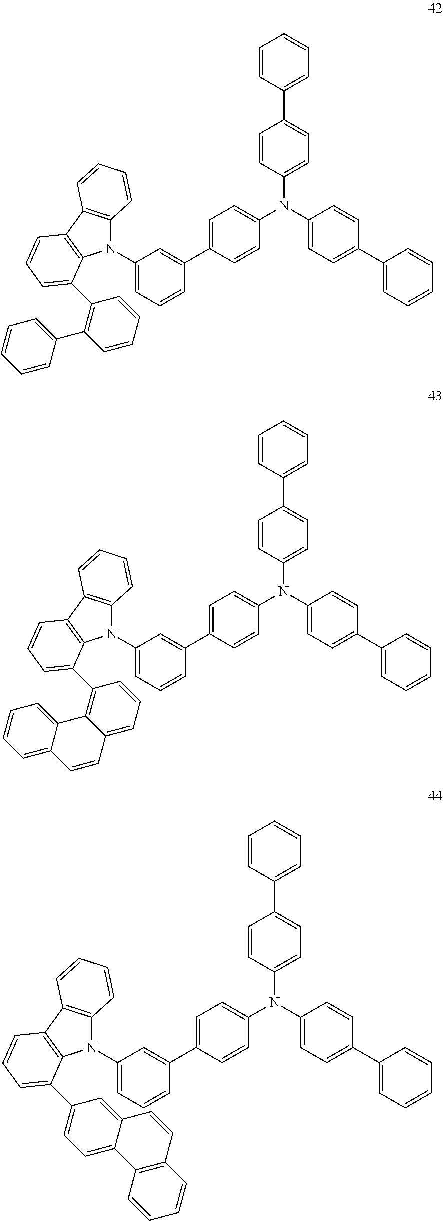 [SMILES: c1ccc(-c2ccc(N(c3ccc(-c4ccccc4)cc3)c3ccc(-c4cccc(-n5c6ccccc6c6cccc(-c7ccccc7-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7cccc8ccc9ccccc9c78)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8c(ccc9ccccc98)c7)c65)c4)cc3)cc2)cc1]

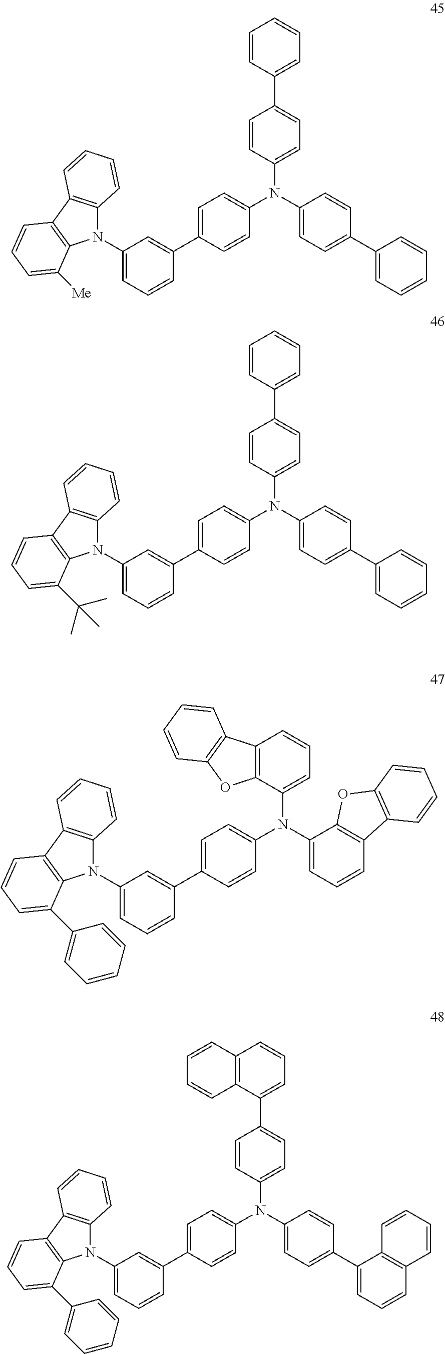 [SMILES: Cc1cccc2c3ccccc3n(-c3cccc(-c4ccc(N(c5ccc(-c6ccccc6)cc5)c5ccc(-c6ccccc6)cc5)cc4)c3)c12 ; CC(C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2cccc3c4ccccc4n(-c4cccc(-c5ccc(N(c6cccc7c6oc6ccccc67)c6cccc7c6oc6ccccc67)cc5)c4)c23)cc1 ; c1ccc(-c2cccc3c4ccccc4n(-c4cccc(-c5ccc(N(c6ccc(-c7cccc8ccccc78)cc6)c6ccc(-c7cccc8ccccc78)cc6)cc5)c4)c23)cc1]

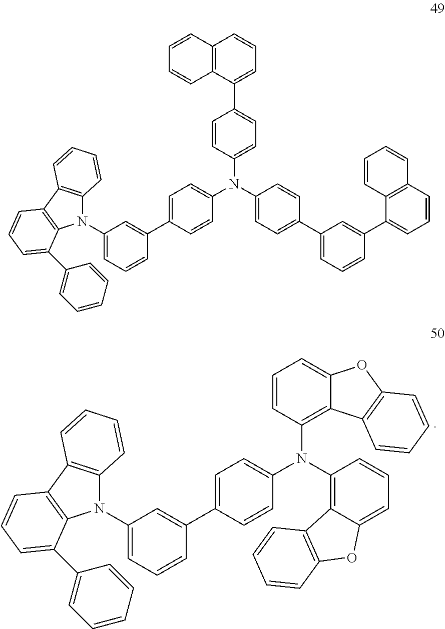 [SMILES: c1ccc(-c2cccc3c4ccccc4n(-c4cccc(-c5ccc(N(c6ccc(-c7cccc(-c8cccc9ccccc89)c7)cc6)c6ccc(-c7cccc8ccccc78)cc6)cc5)c4)c23)cc1 ; c1ccc(-c2cccc3c4ccccc4n(-c4cccc(-c5ccc(N(c6cccc7oc8ccccc8c67)c6cccc7oc8ccccc8c67)cc5)c4)c23)cc1]

7. An organic electroluminescent (EL) device, comprising:
an anode;
a cathode on the anode; and
a plurality of organic layers between the anode and the cathode,
wherein at least one selected from the organic layers comprises a material for an organic EL device, the material being represented by Formula 1: 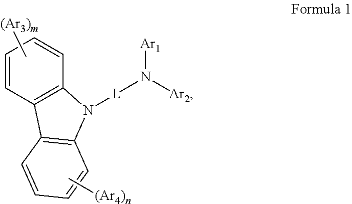
wherein in Formula 1,
Ar₁ and Ar₂ are each independently a substituted or unsubstituted aryl group having 6 to 30 carbon atoms for forming a ring, or a substituted or unsubstituted heteroaryl group having 1 to 30 carbon atoms for forming a ring,
Ar₃ and Ar₄ are each independently a substituted or unsubstituted aryl group having 6 to 30 carbon atoms for forming a ring, a substituted or unsubstituted heteroaryl group having 5 to 30 carbon atoms for forming a ring, an alkyl group having 1 to 6 carbon atoms, or a silyl group,
L is a substituted or unsubstituted arylene group having 6 to 30 carbon atoms for forming a ring, or a substituted or unsubstituted heteroarylene group having 5 to 30 carbon atoms for forming a ring,
m and n are each independently an integer selected from 0 to 4,
m+n≧1, and
at least one of Ar₃ or Ar₄ is substituted in at least one of position 1 or position 8 of a carbazolyl group.

8. The organic EL device of claim 7, further comprising an emission layer between the anode and the cathode,
the material for an organic EL device being comprised in at least one layer between the anode and the emission layer.

9. The organic EL device of claim 7, wherein the organic layer comprising the material for an organic EL device is at least one selected from a hole injection layer and a hole transport layer.

10. The organic EL device of claim 7, wherein in Formula 1, m+n=1.

11. The organic EL device of claim 7, wherein Ar₃ and/or Ar₄ are each independently a phenyl group, a naphthyl group, a biphenyl group, a fluoroaryl group, a dibenzofuranyl group, or a silyl group.

12. The organic EL device of claim 7, wherein L is selected from arylene groups (L-1) to (L-7):

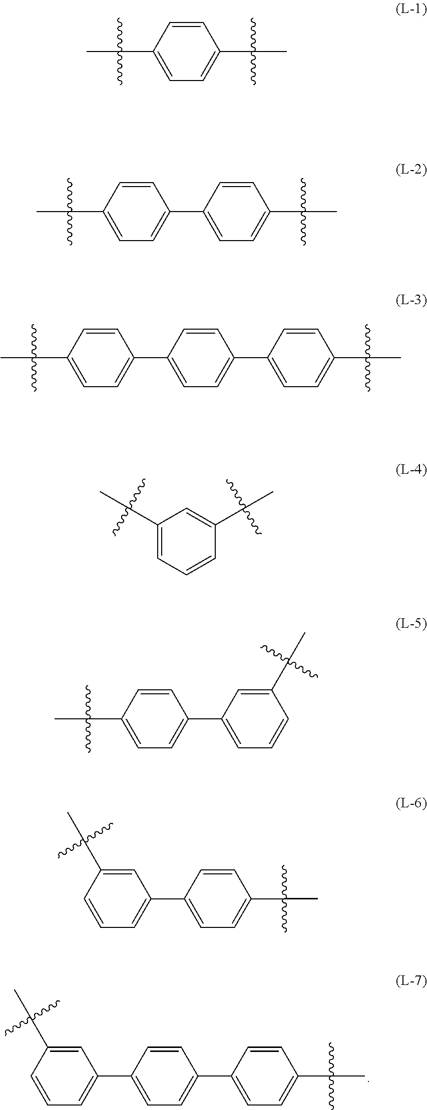

13. The organic EL device of claim 7, wherein L is an m-phenylene group.

14. The organic EL device of claim 7, wherein the material for an organic EL device represented by Formula 1 is represented by at least one selected from Compounds 1 to 50: 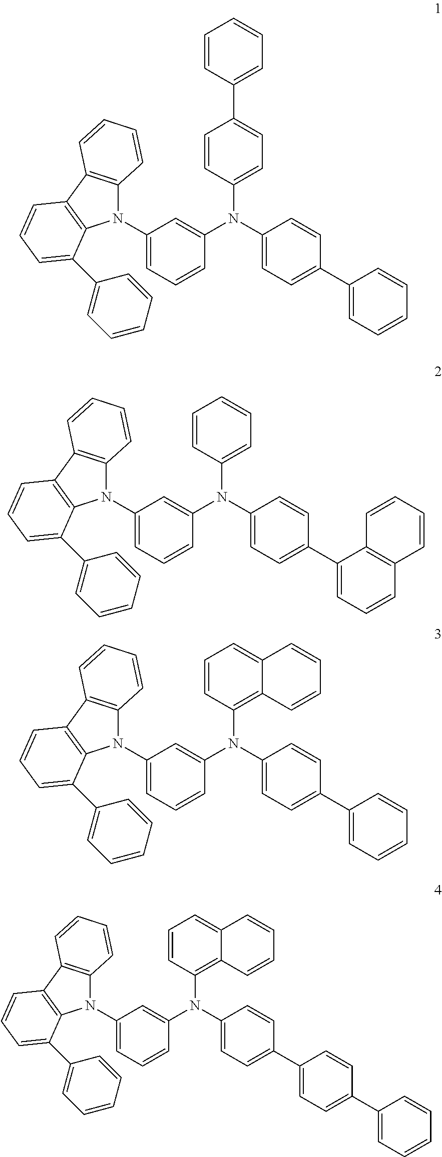 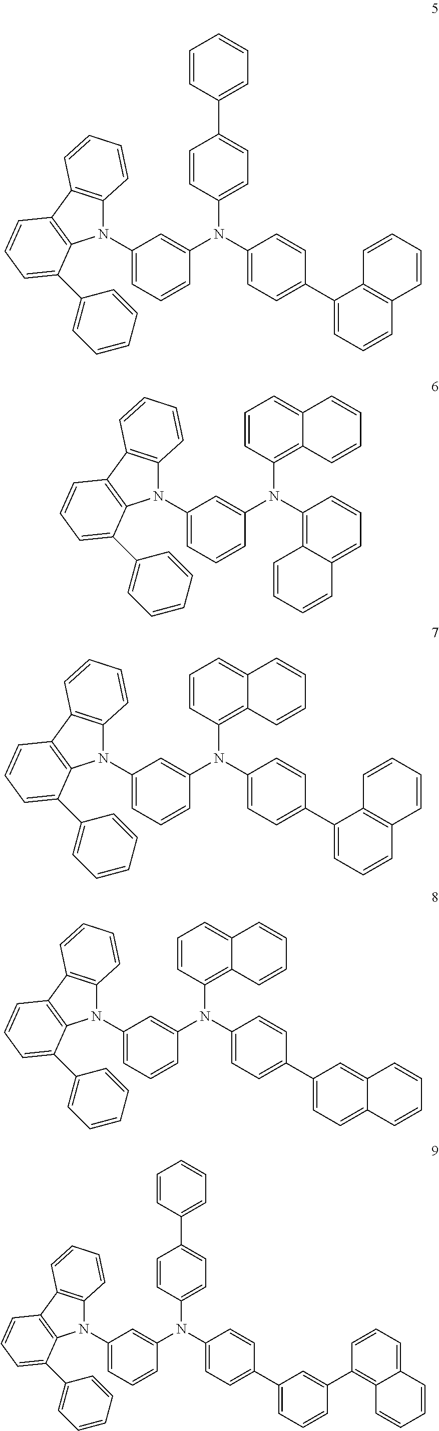 [SMILES: c1ccc(-c2ccc(N(c3ccc(-c4cccc5ccccc45)cc3)c3cccc(-n4c5ccccc5c5cccc(-c6ccccc6)c54)c3)cc2)cc1 ; c1ccc(-c2cccc3c4ccccc4n(-c4cccc(N(c5cccc6ccccc56)c5cccc6ccccc56)c4)c23)cc1 ; c1ccc(-c2cccc3c4ccccc4n(-c4cccc(N(c5ccc(-c6cccc7ccccc67)cc5)c5cccc6ccccc56)c4)c23)cc1 ; c1ccc(-c2cccc3c4ccccc4n(-c4cccc(N(c5ccc(-c6ccc7ccccc7c6)cc5)c5cccc6ccccc56)c4)c23)cc1 ; c1ccc(-c2ccc(N(c3ccc(-c4cccc(-c5cccc6ccccc56)c4)cc3)c3cccc(-n4c5ccccc5c5cccc(-c6ccccc6)c54)c3)cc2)cc1] 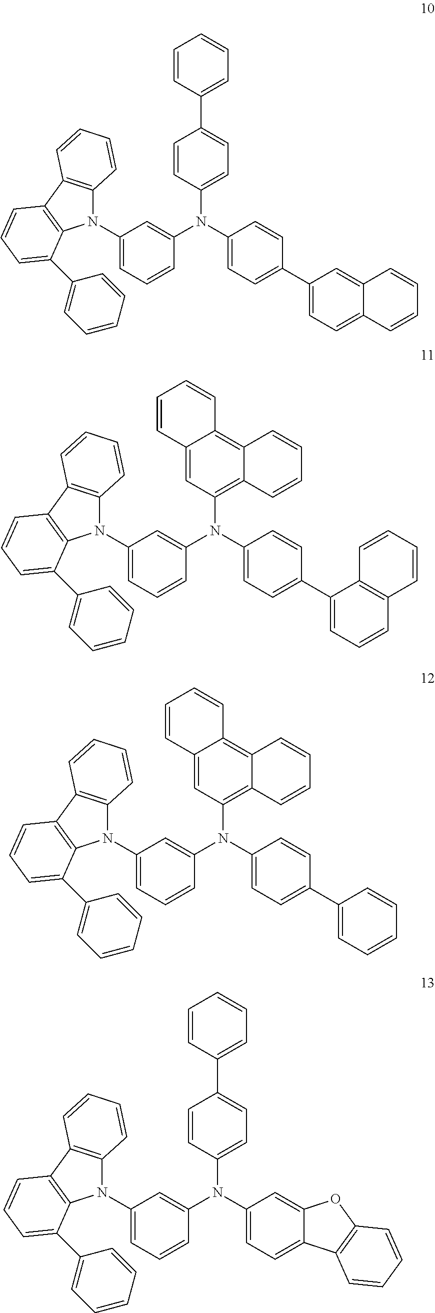 [SMILES: c1ccc(-c2ccc(N(c3ccc(-c4ccc5ccccc5c4)cc3)c3cccc(-n4c5ccccc5c5cccc(-c6ccccc6)c54)c3)cc2)cc1 ; c1ccc(-c2cccc3c4ccccc4n(-c4cccc(N(c5ccc(-c6cccc7ccccc67)cc5)c5cc6ccccc6c6ccccc56)c4)c23)cc1 ; c1ccc(-c2ccc(N(c3cccc(-n4c5ccccc5c5cccc(-c6ccccc6)c54)c3)c3cc4ccccc4c4ccccc34)cc2)cc1 ; c1ccc(-c2ccc(N(c3cccc(-n4c5ccccc5c5cccc(-c6ccccc6)c54)c3)c3ccc4c(c3)oc3ccccc34)cc2)cc1] 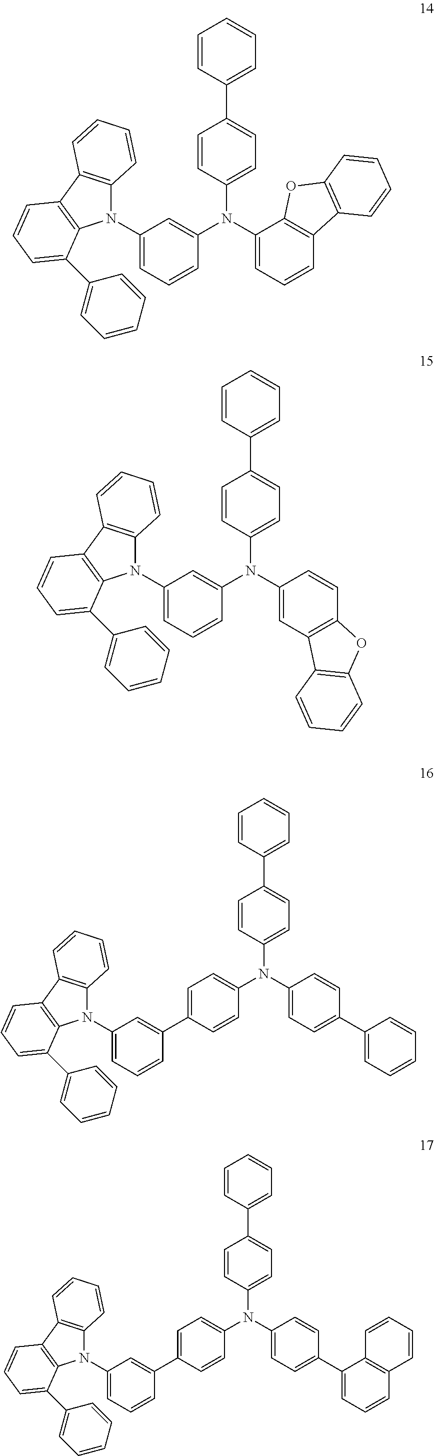 [SMILES: c1ccc(-c2ccc(N(c3cccc(-n4c5ccccc5c5cccc(-c6ccccc6)c54)c3)c3cccc4c3oc3ccccc34)cc2)cc1 ; c1ccc(-c2ccc(N(c3cccc(-n4c5ccccc5c5cccc(-c6ccccc6)c54)c3)c3ccc4oc5ccccc5c4c3)cc2)cc1 ; c1ccc(-c2ccc(N(c3ccc(-c4ccccc4)cc3)c3ccc(-c4cccc(-n5c6ccccc6c6cccc(-c7ccccc7)c65)c4)cc3)cc2)cc1 ; c1ccc(-c2ccc(N(c3ccc(-c4cccc(-n5c6ccccc6c6cccc(-c7ccccc7)c65)c4)cc3)c3ccc(-c4cccc5ccccc45)cc3)cc2)cc1] 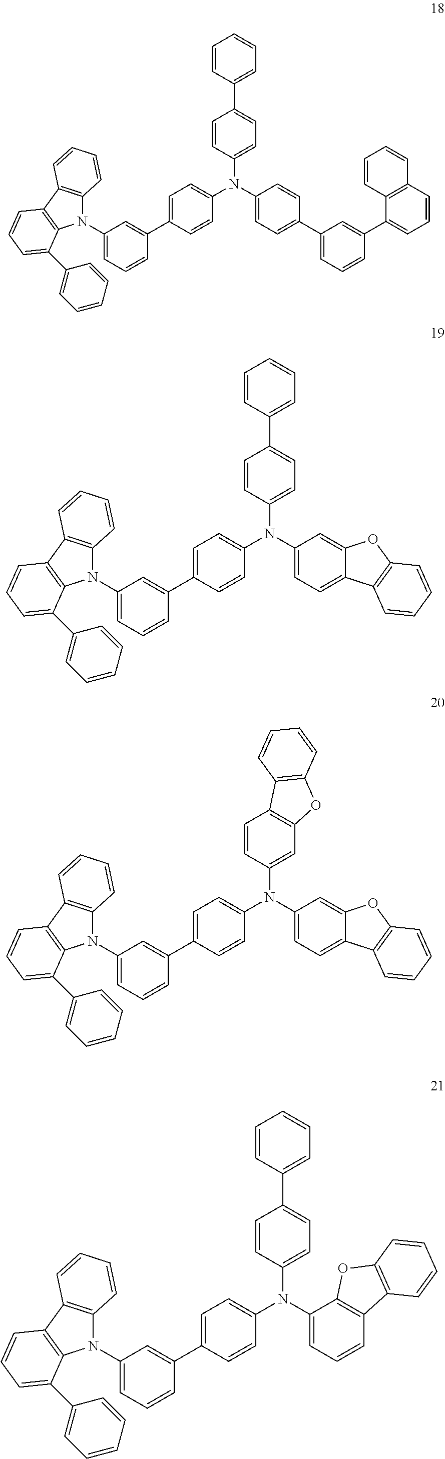 [SMILES: c1ccc(-c2ccc(N(c3ccc(-c4cccc(-c5cccc6ccccc56)c4)cc3)c3ccc(-c4cccc(-n5c6ccccc6c6cccc(-c7ccccc7)c65)c4)cc3)cc2)cc1 ; c1ccc(-c2ccc(N(c3ccc(-c4cccc(-n5c6ccccc6c6cccc(-c7ccccc7)c65)c4)cc3)c3ccc4c(c3)oc3ccccc34)cc2)cc1 ; c1ccc(-c2cccc3c4ccccc4n(-c4cccc(-c5ccc(N(c6ccc7c(c6)oc6ccccc67)c6ccc7c(c6)oc6ccccc67)cc5)c4)c23)cc1 ; c1ccc(-c2ccc(N(c3ccc(-c4cccc(-n5c6ccccc6c6cccc(-c7ccccc7)c65)c4)cc3)c3cccc4c3oc3ccccc34)cc2)cc1] 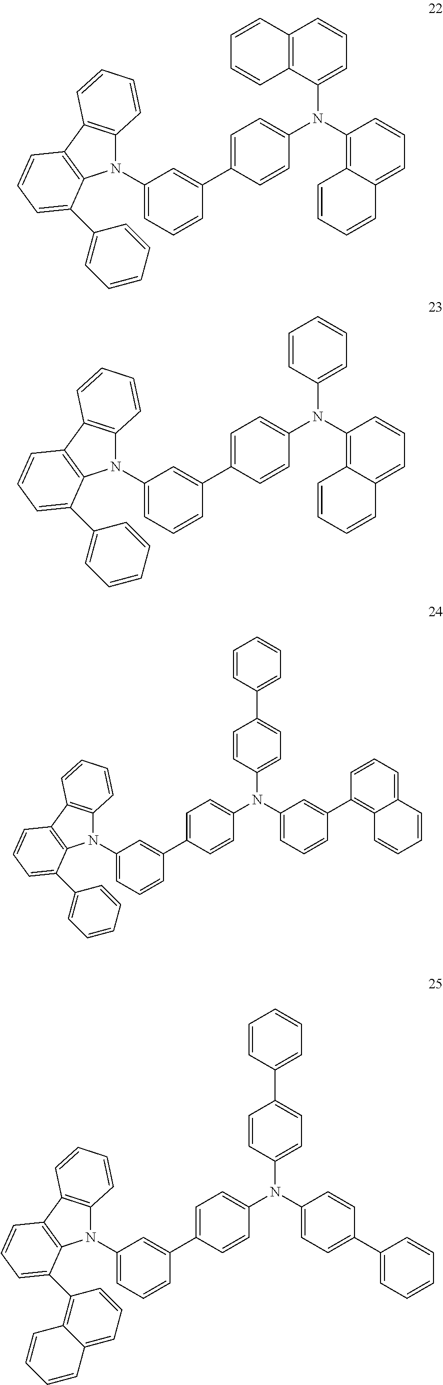 [SMILES: c1ccc(-c2cccc3c4ccccc4n(-c4cccc(-c5ccc(N(c6cccc7ccccc67)c6cccc7ccccc67)cc5)c4)c23)cc1 ; c1ccc(-c2cccc3c4ccccc4n(-c4cccc(-c5ccc(N(c6ccccc6)c6cccc7ccccc67)cc5)c4)c23)cc1 ; c1ccc(-c2ccc(N(c3ccc(-c4cccc(-n5c6ccccc6c6cccc(-c7ccccc7)c65)c4)cc3)c3cccc(-c4cccc5ccccc45)c3)cc2)cc1 ; c1ccc(-c2ccc(N(c3ccc(-c4ccccc4)cc3)c3ccc(-c4cccc(-n5c6ccccc6c6cccc(-c7cccc8ccccc78)c65)c4)cc3)cc2)cc1] 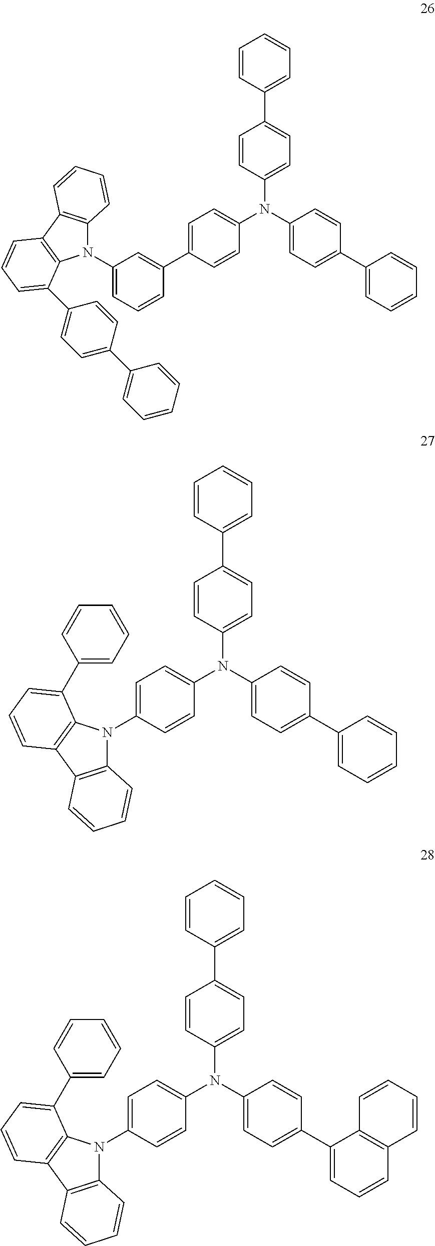 [SMILES: c1ccc(-c2ccc(-c3cccc4c5ccccc5n(-c5cccc(-c6ccc(N(c7ccc(-c8ccccc8)cc7)c7ccc(-c8ccccc8)cc7)cc6)c5)c34)cc2)cc1 ; c1ccc(-c2ccc(N(c3ccc(-c4ccccc4)cc3)c3ccc(-n4c5ccccc5c5cccc(-c6ccccc6)c54)cc3)cc2)cc1 ; c1ccc(-c2ccc(N(c3ccc(-c4cccc5ccccc45)cc3)c3ccc(-n4c5ccccc5c5cccc(-c6ccccc6)c54)cc3)cc2)cc1] 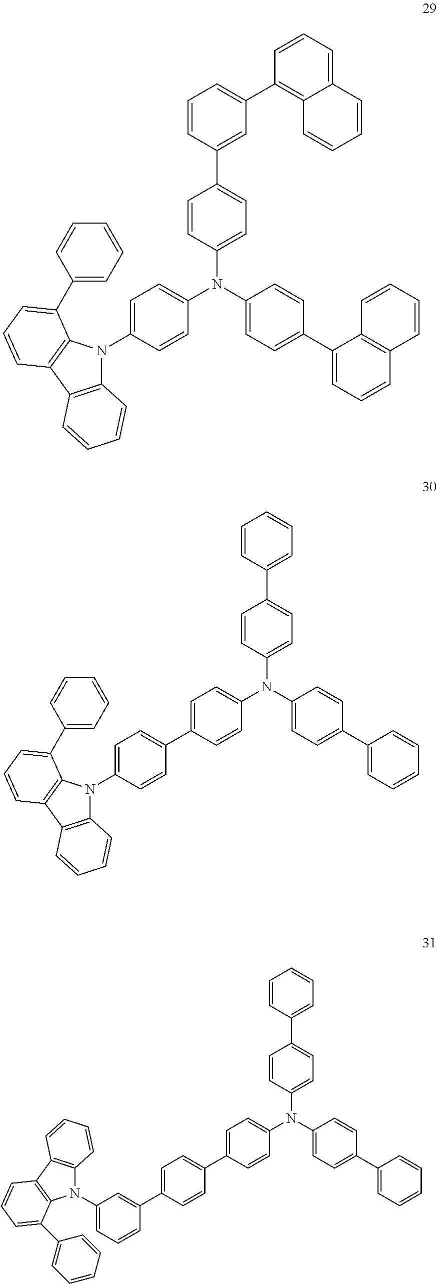 [SMILES: c1ccc(-c2cccc3c4ccccc4n(-c4ccc(N(c5ccc(-c6cccc(-c7cccc8ccccc78)c6)cc5)c5ccc(-c6cccc7ccccc67)cc5)cc4)c23)cc1 ; c1ccc(-c2ccc(N(c3ccc(-c4ccccc4)cc3)c3ccc(-c4ccc(-n5c6ccccc6c6cccc(-c7ccccc7)c65)cc4)cc3)cc2)cc1 ; c1ccc(-c2ccc(N(c3ccc(-c4ccccc4)cc3)c3ccc(-c4ccc(-c5cccc(-n6c7ccccc7c7cccc(-c8ccccc8)c76)c5)cc4)cc3)cc2)cc1] 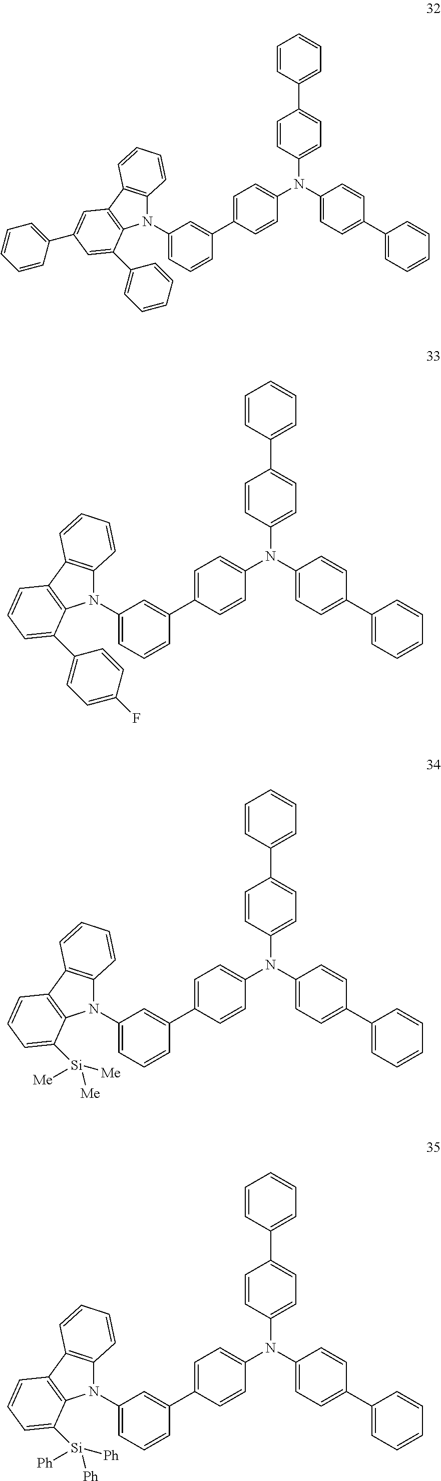 [SMILES: c1ccc(-c2ccc(N(c3ccc(-c4ccccc4)cc3)c3ccc(-c4cccc(-n5c6ccccc6c6cc(-c7ccccc7)cc(-c7ccccc7)c65)c4)cc3)cc2)cc1 ; Fc1ccc(-c2cccc3c4ccccc4n(-c4cccc(-c5ccc(N(c6ccc(-c7ccccc7)cc6)c6ccc(-c7ccccc7)cc6)cc5)c4)c23)cc1 ; C[Si](C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2ccc(N(c3ccc(-c4ccccc4)cc3)c3ccc(-c4cccc(-n5c6ccccc6c6cccc([Si](c7ccccc7)(c7ccccc7)c7ccccc7)c65)c4)cc3)cc2)cc1] 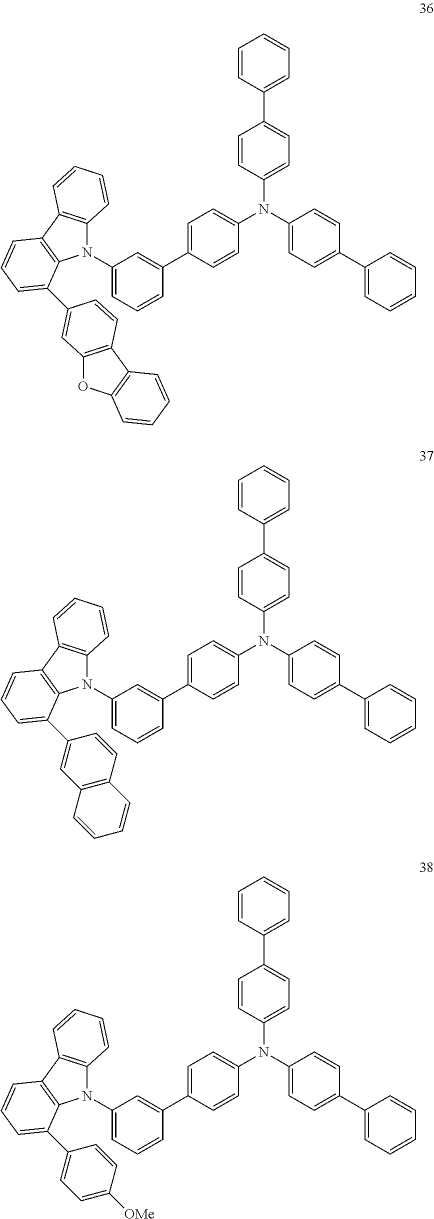 [SMILES: c1ccc(-c2ccc(N(c3ccc(-c4ccccc4)cc3)c3ccc(-c4cccc(-n5c6ccccc6c6cccc(-c7ccc8c(c7)oc7ccccc78)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8ccccc8c7)c65)c4)cc3)cc2)cc1 ; COc1ccc(-c2cccc3c4ccccc4n(-c4cccc(-c5ccc(N(c6ccc(-c7ccccc7)cc6)c6ccc(-c7ccccc7)cc6)cc5)c4)c23)cc1] 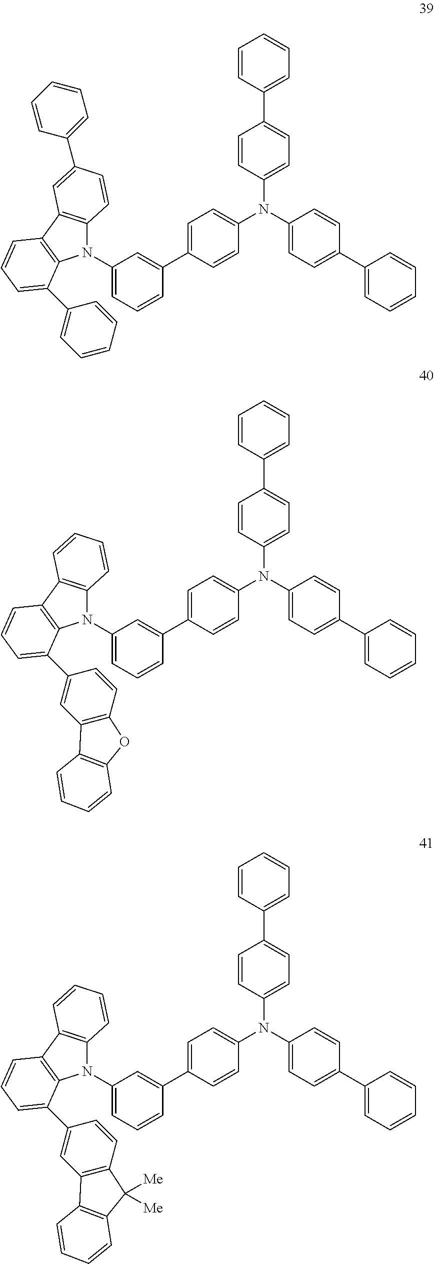 [SMILES: c1ccc(-c2ccc(N(c3ccc(-c4ccccc4)cc3)c3ccc(-c4cccc(-n5c6ccc(-c7ccccc7)cc6c6cccc(-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8oc9ccccc9c8c7)c65)c4)cc3)cc2)cc1 ; CC1(C)c2ccccc2-c2cc(-c3cccc4c5ccccc5n(-c5cccc(-c6ccc(N(c7ccc(-c8ccccc8)cc7)c7ccc(-c8ccccc8)cc7)cc6)c5)c34)ccc21] 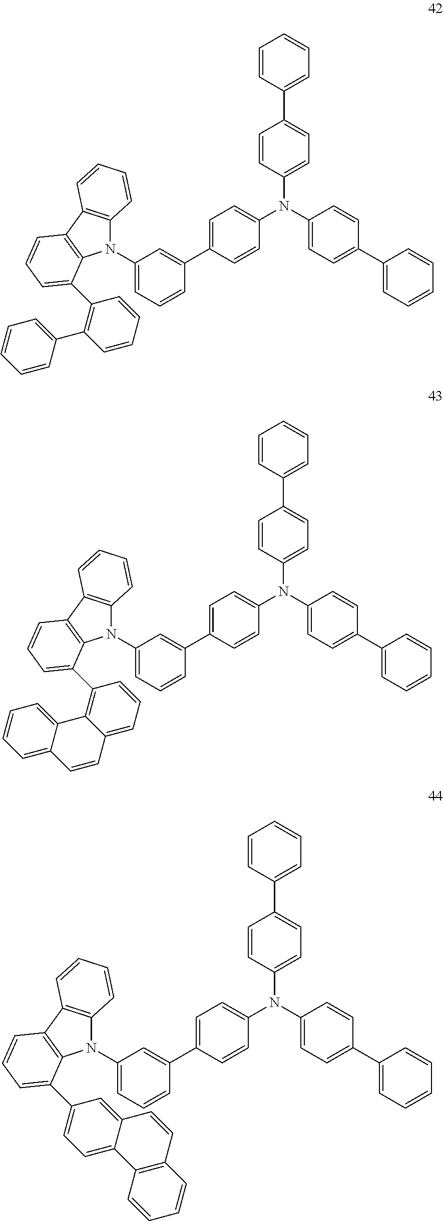 [SMILES: c1ccc(-c2ccc(N(c3ccc(-c4ccccc4)cc3)c3ccc(-c4cccc(-n5c6ccccc6c6cccc(-c7ccccc7-c7ccccc7)c65)c4)cc3)cc2)cc1 ; c1ccc(-c2ccc(N(c3ccc(-c4ccccc4)cc3)c3ccc(-c4cccc(-n5c6ccccc6c6cccc(-c7cccc8ccc9ccccc9c78)c65)c4)cc3)cc2)cc1 ; c1ccc(-c2ccc(N(c3ccc(-c4ccccc4)cc3)c3ccc(-c4cccc(-n5c6ccccc6c6cccc(-c7ccc8c(ccc9ccccc98)c7)c65)c4)cc3)cc2)cc1] 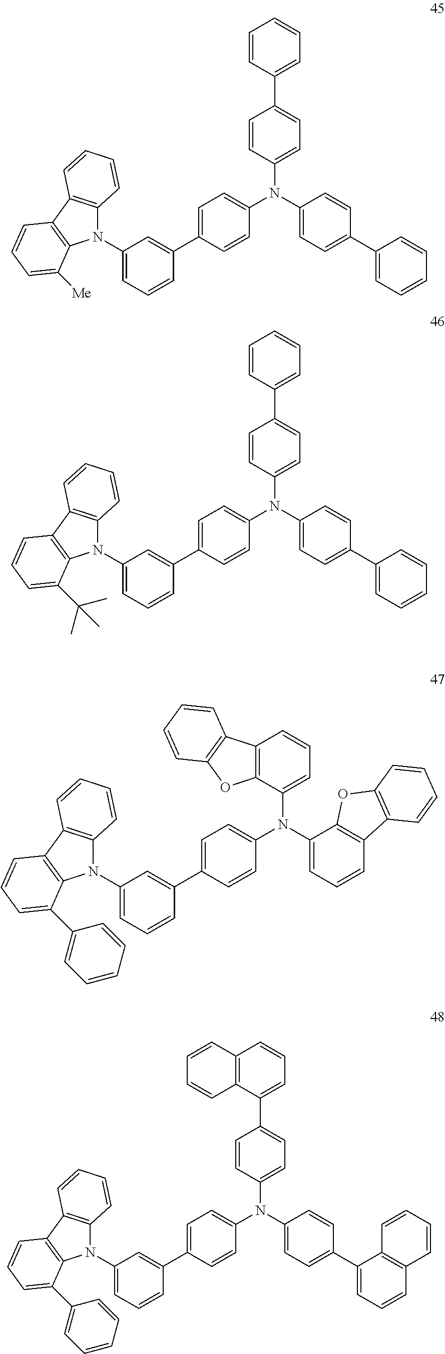 [SMILES: Cc1cccc2c3ccccc3n(-c3cccc(-c4ccc(N(c5ccc(-c6ccccc6)cc5)c5ccc(-c6ccccc6)cc5)cc4)c3)c12 ; CC(C)(C)c1cccc2c3ccccc3n(-c3cccc(-c4ccc(N(c5ccc(-c6ccccc6)cc5)c5ccc(-c6ccccc6)cc5)cc4)c3)c12 ; c1ccc(-c2cccc3c4ccccc4n(-c4cccc(-c5ccc(N(c6cccc7c6oc6ccccc67)c6cccc7c6oc6ccccc67)cc5)c4)c23)cc1 ; c1ccc(-c2cccc3c4ccccc4n(-c4cccc(-c5ccc(N(c6ccc(-c7cccc8ccccc78)cc6)c6ccc(-c7cccc8ccccc78)cc6)cc5)c4)c23)cc1] 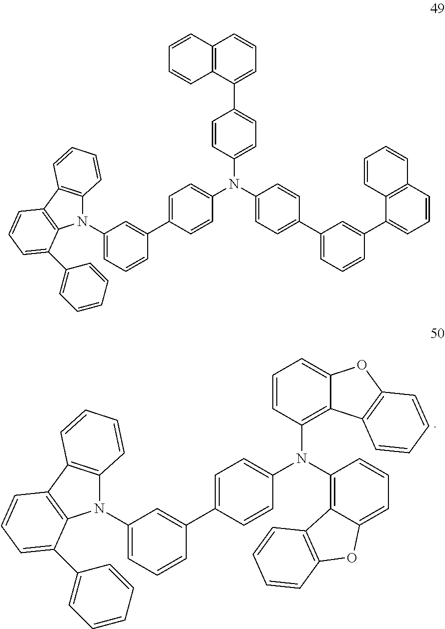 [SMILES: c1ccc(-c2cccc3c4ccccc4n(-c4cccc(-c5ccc(N(c6ccc(-c7cccc(-c8cccc9ccccc89)c7)cc6)c6ccc(-c7cccc8ccccc78)cc6)cc5)c4)c23)cc1 ; c1ccc(-c2cccc3c4ccccc4n(-c4cccc(-c5ccc(N(c6cccc7oc8ccccc8c67)c6cccc7oc8ccccc8c67)cc5)c4)c23)cc1]
